(19) United States
(12) Patent Application Publication
(10) Pub. No.: US 2026/0182881 A1
(43) Pub. Date: Jul. 2, 2026

(54) EMOTIONAL STATE ANALYSIS AND REAL-TIME FITNESS COACHING

(71) Applicant: Meta Platforms, Inc., Menlo Park, CA (US)

(72) Inventor: Lachlan Dunn, Los Angeles, CA (US)

(21) Appl. No.: 19/422,320

(22) Filed: Dec. 16, 2025

Related U.S. Application Data
(60) Provisional application No. 63/738,984, filed on Dec. 26, 2024.

(51) Int. Cl. A61B 5/16; A61B 5/00; A63B 24/00; A63B 71/06

(52) U.S. Cl. CPC A61B 5/165; A61B 5/4803; A61B 5/7246; A61B 5/7267; A63B 24/0075; A63B 71/0622; A63B 2071/0625; A63B 2071/0666; A63B 2220/806

(57) ABSTRACT

Systems and methods for analyzing an emotional state of a user over time include receiving audible communications of the user across one or more contexts. The audible communications may be associated with contextual factors such as time of day, location, user activity, or digital interaction. The audible communications may be transcribed, and an emotional-state machine learning model may interpret verbal and nonverbal cues to determine emotional indicators. The emotional indicators may be correlated with the contextual factors to generate correlated emotional indicators. Portions of the audible communications associated with the correlated emotional indicators may be determined as citations. A summary of emotional trends of the user may be generated over the period of time based on the correlated emotional indicators. The summary may include the citations.

### CROSS-REFERENCE TO RELATED APPLICATION

The present application claims the benefit of U.S. Provisional Patent Application No. 63/738,984, filed on Dec. 26, 2024, and titled “METHODS, APPARATUSES, SYSTEMS AND COMPUTER PROGRAM PRODUCTS FOR EMOTIONAL STATE ANALYSIS AND REAL-TIME FITNESS COACHING,” the disclosure of which is expressly incorporated by reference in its entirety.

### TECHNOLOGICAL FIELD

Exemplary embodiments of this disclosure relate generally to methods, apparatuses, and computer programs for artificial intelligence providing emotional state analysis and real-time fitness coaching.

### BACKGROUND

Advances in machine learning, multimodal sensing, and consumer-grade wearable devices have enabled individuals to better understand their daily behaviors, physiological states, and overall well-being. Modern communication devices, such as smart glasses, smartphones, smart home speakers, wearable headsets, and fitness-oriented wearables, may incorporate microphones, cameras, inertial sensors, and biometric sensors that continuously detect, record, and interpret a wide variety of user inputs. These inputs can include audible utterances, speech patterns, nonverbal vocalizations, visual cues, body movements, and/or other real-time signals available to the device.

Additionally, artificial intelligence (AI) systems have become increasingly capable of identifying sentiment, inferring affective characteristics, classifying exercises, detecting body poses, and analyzing multimodal data streams with high accuracy. Machine learning models, including large language models, multimodal speech models, and pose-estimation frameworks, now support real-time inference on both lightweight consumer devices and remote servers. These systems are capable of learning from large collections of audio, video, and behavioral data, and can be adapted to specific users over time by incorporating user-specific patterns, signals, and contextual information.

Wearable devices and augmented/virtual-reality systems may be integrated into daily routines to enable continuous or intermittent capture of audio, video, and motion data. Smart glasses, for example, may include forward-facing cameras, eye-tracking sensors, microphones, and display elements, enabling hands-free capture of environmental context, user interactions, and user behavior throughout the day. Similarly, fitness applications increasingly leverage multimodal data such as high-frequency IMU readings, skeletal pose estimation, and video-based motion analysis to deliver exercise guidance or generate customized workout routines.

### BRIEF SUMMARY

Systems and methods to facilitate artificial intelligence providing emotional state analysis and real-time fitness coaching are provided. The systems and methods may enable the use of artificial intelligence to perform sentiment analysis on one or more recorded audible communications to generate a summary of a user's emotional state over time. The systems and methods may further enable the use of artificial intelligence to generate a workout routine or observe a user's exercises to provide guidance on exercise form(s) and technique(s). In some example aspects, the methods, systems, and apparatuses may facilitate monitoring audio communications and providing a summary of the emotional states of a user(s) over time.

Some aspects of the present disclosure are directed to analyzing an emotional state of a user over time based on audible communications and contextual information. In some aspects, a method includes receiving audible communications of a user over a period of time, generating a transcript of the audible communications, and determining one or more emotional indicators using an emotional-state machine learning model configured to interpret verbal and nonverbal cues. The method further includes correlating the emotional indicators with contextual factors such as time of day, location, user activity, or digital interaction to generate correlated emotional indicators. Portions of the audible communications associated with the correlated emotional indicators may be determined as citations, and a summary of emotional trends may be generated based on the correlated emotional indicators. The summary may include the citations.

In another example of the present disclosure, an apparatus is provided. The apparatus may include one or more processors and a memory including computer program code instructions. The memory and computer program code instructions are configured to, with at least one of the processors, cause the apparatus to at least perform operations including receiving, over a period of time, an audible communication of a user. The audible communication associated with a contextual factor comprising a time of day, a location, an activity of the user, or a digital interaction of the user. The memory and computer program code are also configured to, with the processor(s), cause the apparatus to generate a transcript associated with the audible communication. The memory and computer program code are also configured to, with the processor(s), cause the apparatus to determine, via an emotional-state machine learning model configured to interpret verbal and nonverbal cues of the user, an emotional indicator associated with the audible communication. The memory and computer program code are also configured to, with the processor(s), cause the apparatus to generate correlated emotional indicators based on correlating the emotional indicator with the contextual factor. The memory and computer program code are also configured to, with the processor(s), cause the apparatus to determine, based on the transcript and the correlated emotional indicators, a citation that reference portions of the audible communication associated with the correlated emotional indicators. The memory and computer program code are also configured to, with the processor(s), cause the apparatus to generate a summary of emotional trends of the user over the period of time based on the correlated emotional indicators. The summary of emotional trends may comprise the citation.

In yet another example of the present disclosure, a computer program product is provided. The computer program product may include at least one non-transitory computer-readable medium including computer-executable program code instructions stored therein. The computer-executable program code instructions may include program code instructions configured to receive, over a period of time, an audible communication of a user. The audible communication associated with a contextual factor comprising a time of day, a location, an activity of the user, or a digital interaction of the user. The computer program product may further include program code instructions configured to generate a transcript associated with the audible communication. The computer program product may further include program code instructions configured to determine, via an emotional-state machine learning model configured to interpret verbal and nonverbal cues of the user, an emotional indicator associated with the audible communication. The computer program product may further include program code instructions configured to generate correlated emotional indicators based on correlating the emotional indicator with the contextual factor. The computer program product may further include program code instructions configured to determine, based on the transcript and the correlated emotional indicators, a citation that reference portions of the audible communication associated with the correlated emotional indicators. The computer program product may further include program code instructions configured to generate a summary of emotional trends of the user over the period of time based on the correlated emotional indicators. The summary of emotional trends may comprise the citation.

In other aspects, an apparatus includes one or more processors and one or more memories storing instructions that, when executed by the one or more processors, cause the apparatus to receive audible communications, generate transcripts, determine emotional indicators, correlate the emotional indicators with contextual factors, identify citations associated with the emotional indicators, and generate a summary of emotional trends of the user over the period of time.

In further aspects, a non-transitory computer-readable medium stores program code, the program code may be executed by one or more processors and includes program code to perform operations for emotional state analysis, including program code to receive audible communications associated with contextual factors, program code to generate transcripts, program code to determine emotional indicators using a machine learning model, program code to generate correlated emotional indicators, program code to determine citations to portions of the audible communications, and program code to generate a summary of emotional trends comprising the citations.

Some aspects of the present disclosure further relate to techniques for providing fitness coaching that adapts to a user's emotional state. In some aspects, a method includes receiving a workout preference from a user, generating a workout instruction based on the workout preference and workout examples stored in a training database, and receiving image data depicting movements of the user during a workout session via a camera of a device worn by the user. The method further includes adjusting the workout instruction based on a summary of emotional trends associated with the user, such that the workout instruction reflects both physical performance and emotional condition.

In other aspects, an apparatus includes one or more processors and one or more memories storing instructions that, when executed by the one or more processors, cause the apparatus to receive workout preferences, generate workout instructions, receive image data depicting user movements during a workout session, and adjust the workout instructions based on emotional trend information associated with the user.

In further aspects, a non-transitory computer-readable medium stores program code. The program code may be executed by one or more processors and includes program code to perform operations for emotion-adaptive fitness coaching. The program code includes program code to receive a workout preference from a user, program code to generate workout instructions based on the workout preference and workout examples stored in a training database, program code to receive image data depicting movements of the user during a workout session via one or more cameras, program code to analyze the image data to determine movement characteristics of the user, and program code to modify the workout instructions based on a summary of emotional trends derived from emotional state analysis.

An AI assistant may be utilized in accordance with exemplary aspects of the present disclosure to listen to oral communication and/or provide insight into a user and/or the user's emotional state. For example, the AI assistant may listen to a user(s) at predefined times to hear various types of communication, such as sighs, laughter, and/or the tone(s) of a voice(s). The AI assistant may use these inputs to quantify the user's emotional state or generate other insights about the user. In an example, the AI assistant may track emotion over a predetermined time period (e.g., a month, etc.) and/or provide a summary of emotional states at the end/expiration of the predetermined time period (e.g., the month). The AI assistant may be able to provide a summary of emotional state or mood at different times of a day and/or different parts of a time period (e.g., the month). In another example, the AI assistant may take multiple inputs in addition to audio inputs (e.g., of a user's voice) to provide a summary of emotional trends based on various inputs (e.g., a happier emotional state associated with a particular time of day or at a time when medication is taken, etc.).

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### DESCRIPTION OF DRAWINGS

The summary, as well as the following detailed description, is further understood when read in conjunction with the appended drawings. For the purpose of illustrating the disclosed subject matter, there are shown in the drawings exemplary embodiments of the disclosed subject matter; however, the disclosed subject matter is not limited to the specific methods, compositions, and devices disclosed. In addition, the drawings are not necessarily drawn to scale. In the drawings:

FIG. 1 is a diagram of an exemplary network environment, in accordance with an example of the present disclosure.

FIG. 2 is a diagram of an exemplary communication device, in accordance with an example of the present disclosure.

FIG. 3 is a diagram of an example computing system, in accordance with an example of the present disclosure.

FIG. 4 illustrates an example of an artificial reality system, in accordance with various aspects of the present disclosure.

FIG. 5 illustrates an example of an artificial reality system, in accordance with various aspects of the present disclosure.

FIG. 6 illustrates an example system to facilitate emotional state analysis, in accordance with various aspects of the present disclosure.

FIG. 7 illustrates an example of a system that facilitates real-time coaching using artificial intelligence, in accordance with various aspects of the present disclosure.

FIG. 8 illustrates an example of a system that facilitates real-time coaching using artificial intelligence, in accordance with various aspects of the present disclosure.

FIG. 9 illustrates an example of a machine learning framework including machine learning model(s) and a training database, in accordance with various aspects of the present disclosure.

FIG. 10 is a flow diagram illustrating an example of a process for analyzing an emotional state, in accordance with various aspects of the present disclosure.

FIG. 11 is a flow diagram illustrating an example of a process for real-time fitness coaching, in accordance with various aspects of the present disclosure.

The figures depict various embodiments for illustrative purposes only. One skilled in the art will readily recognize from the following discussion that alternative embodiments of the structures and methods illustrated herein may be employed without departing from the principles described herein.

### DETAILED DESCRIPTION

Some embodiments of the present invention will now be described more fully hereinafter with reference to the accompanying drawings, in which some, but not all embodiments of the invention are shown. Various embodiments of the invention may be embodied in many different forms and should not be construed as limited to the embodiments set forth herein. Like reference numerals refer to like elements throughout.

It is to be understood that the methods and systems described herein are not limited to specific methods, specific components, or to particular implementations. It is also to be understood that the terminology used herein is for the purpose of describing particular embodiments only and is not intended to be limiting.

Herein, a computer-readable non-transitory storage medium or media may include one or more semiconductor-based or other integrated circuits (ICs) (such as, for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, computer readable medium or any suitable combination of two or more of these, where appropriate. A computer-readable non-transitory storage medium may be volatile, non-volatile, or a combination of volatile and non-volatile, where appropriate.

As defined herein, a “computer-readable storage medium,” which refers to a non-transitory, physical or tangible storage medium (e.g., volatile or non-volatile memory device), may be differentiated from a “computer-readable transmission medium,” which refers to an electromagnetic signal.

As referred to herein, a Metaverse may denote an immersive virtual space or world in which devices may be utilized in a network in which there may, but need not, be one or more social connections among users in the network or with an environment in the virtual space or world. A Metaverse or Metaverse network may be associated with three-dimensional (3D) virtual worlds, online games (e.g., video games), one or more content items such as, for example, images, videos, non-fungible tokens (NFTs) and in which the content items may, for example, be purchased with digital currencies (e.g., cryptocurrencies) and other suitable currencies. In some examples, a Metaverse or Metaverse network may enable the generation and provision of immersive virtual spaces in which remote users may socialize, collaborate, learn, shop, and/or engage in various other activities within the virtual spaces, including through the use of Augmented/Virtual/Mixed Reality.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

Also, as used in the specification, including the appended claims, the singular forms “a,” “an,” and “the” include the plural, and a reference to a particular numerical value includes at least that value, unless the context clearly dictates otherwise. The term “plurality”, as used herein, means more than one. When a range of values is expressed, another embodiment includes from one particular value or to the other particular value. Similarly, when values are expressed as approximations, by use of the antecedent “about,” it will be understood that the particular value forms another embodiment. All ranges are inclusive and combinable. It is to be understood that the terminology used herein is for the purpose of describing particular aspects only and is not intended to be limiting.

This written description uses examples to enable any person skilled in the art to practice the claimed subject matter, including making and using any devices or systems and performing any incorporated methods. Other variations of the examples are contemplated herein. It is to be appreciated that certain features of the disclosed subject matter which are, for clarity, described herein in the context of separate embodiments, may also be provided in combination in a single embodiment. Conversely, various features of the disclosed subject matter that are, for brevity, described in the context of a single embodiment, may also be provided separately or in any sub-combination. Further, any reference to values stated in ranges includes each and every value within that range. Any documents cited herein are incorporated herein by reference in their entirety for any and all purposes.

The scope of this disclosure encompasses all changes, substitutions, variations, alterations, and modifications to the example embodiments described or illustrated herein that a person having ordinary skill in the art would comprehend. The scope of this disclosure is not limited to the examples described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

As discussed, intelligence, multimodal sensing, and consumer devices continue to evolve, giving users new ways to understand their behavior throughout the day. Modern devices such as smartphones, smart home speakers, smart glasses, wearable fitness trackers, and lightweight headsets collect audio, video, motion, and biometric information through integrated sensors. Machine learning models interpret these inputs, identify emotional signals, recognize speech patterns, classify body movements, and evaluate physical activity with increasing accuracy.

Wearable and augmented reality devices also give users continuous access to sensing and feedback capabilities during daily routines. These devices combine microphones, cameras, motion sensors, and on-board processors with large-scale machine learning models that operate locally or through connected systems. As these technologies improve, developers continue to build systems that gather multimodal user inputs, analyze behavior in real time, and deliver personalized insights that reflect each user's unique patterns and context.

Current tools for emotional wellness and physical fitness leave significant gaps that AI-enabled systems can address. For example, existing emotional wellness tools may rely on self-reporting or short, isolated interactions. Users record their feelings only when they remember to do so, which prevents a continuous, objective view of daily emotional patterns. These tools usually focus on direct verbal responses and ignore nonverbal cues such as sighs, laughter, changes in tone of voice, or spontaneous utterances. Users who rely on these tools never receive a complete picture of their emotional shifts throughout the day or across different environments.

Additionally, fitness technologies require multiple sensors, fixed cameras, or heavy headsets that restrict natural movement. Users often need to purchase extra equipment or set up a specific workout space. These requirements increase friction and reduce accessibility. When users start exercising, these systems often struggle to deliver real-time, precise feedback. Users may receive only general guidance on technique rather than accurate information that reflects their exact body position, movement quality, or form.

Various aspects of the present disclosure are directed to monitoring emotional signals, tracking emotional trends, generating tailored workout routines, and delivering real-time fitness coaching. These aspects leverage multimodal inputs, integrated device hardware, and advanced machine learning models to provide each user with personalized insights and guidance throughout the day. In some examples, an emotional state analysis component records audio from one or more user devices, such as smart glasses, smartphones, smartwatches, mobile tablets, laptops, headphones, microphones, and/or smart home speakers. These devices capture spoken words, changes in tone, spontaneous sighs, laughter, and other nonverbal cues that reflect emotional expression. Some implementations transcribe recorded audio and evaluate the transcriptions using AI models, including large language models trained for sentiment analysis. These models interpret emotional cues in both verbal and nonverbal sounds and generate emotional indicators that reflect the user's reactions and mood throughout the day. Certain implementations use these indicators to establish an emotional baseline and then monitor the user at preselected times to identify changes or emerging emotional patterns. Other implementations evaluate additional information such as biometric readings, eye movement indicators, pupil dilation, respiration signals, and digital activity behavior to correlate emotional state with contextual factors that do not depend on speech. These techniques can identify relationships between emotional fluctuations and daily habits, such as improved mood after medication, increased sighing between meetings, or consistent reactions during specific time periods. Aspects of the disclosure aggregate these multimodal signals across selected intervals and produce summaries that present emotional trends, highlight key moments, and display correlations that help the user understand long term emotional behavior. Devices that support visual interfaces or audio feedback present these emotional insights to the user through displays or spoken output.

In some examples, such aspects introduce a multimodal audio processing pipeline that operates continuously across lightweight devices. For example, a system may record audio signals, detect nonverbal cues, and interpret emotional tone with machine learning models that optimize both latency and accuracy on resource-constrained hardware. The system may increase efficiency by performing on-device transcription, incremental emotion scoring, and real-time trend aggregation, which reduces the need for large server requests. The system also improves the technical field of speech analysis by fusing audio features with biometric signals, eye movement data, and digital activity inputs to produce an emotional profile that relies on cross-modal correlations rather than single-stream prediction. The system increases the precision and reliability of emotional inference by aligning multimodal sensor inputs on synchronized timelines, which creates a novel data structure that supports richer emotional analysis. These combined features deliver a technical improvement in automated audio interpretation, enabling continuous emotional monitoring on everyday devices.

In some examples, various aspects of the present disclosure are directed to a real-time fitness coaching system that observes body movements through cameras and motion sensors on smart glasses, mobile devices, or other computing devices. The system analyzes body pose, joint angles, and/or movement quality using machine learning models, then delivers precise corrective feedback via audio and/or visual cues. The system guides the user through exercises with detailed form adjustments, position indicators, and repetition tracking that reflect the user's real-time performance.

The real-time fitness coaching system improves computer technology by coordinating visual and motion sensors to generate precise pose evaluations. The system processes camera feeds, mirror reflections, and/or inertial measurements in a single multimodal model that identifies skeletal positions with improved accuracy. The system advances pose estimation techniques by aligning inertial sensor data with visual landmarks to correct drift, resolve occlusions, and/or stabilize pose predictions during fast or complex movements. The system increases the reliability of real-time feedback by computing joint angles, spatial alignment, and/or movement vectors on device hardware without perceptible delay. The system also produces a technical improvement in exercise recognition because it detects small deviations in form that conventional computer vision models cannot identify. The system supplies corrective instructions that depend on numerical thresholds that the system calculates from its multimodal signals, which enhances the precision and responsiveness of digital fitness coaching.

In some examples, the emotional analysis may be integrated with the fitness coaching. In such examples, emotional indicators such as mood patterns, stress signals, and/or energy levels may be used to adjust workout intensity, exercise selection, and/or rest intervals. The system aligns the user's emotional readiness with the physical guidance provided during each workout, creating a unified experience that supports both emotional wellness and physical performance. A human cannot calculate these numerical thresholds or track micro deviations in real time with the same precision or consistency. As a result, the system provides a level of corrective feedback that surpasses human capability, thereby improving the technical performance of digital fitness coaching.

The integrated emotional and fitness system improves computer technology by combining emotional state analysis and physical movement evaluation within a single adaptive control framework. The system synchronizes emotional indicators, biometric signals, and exercise data to generate dynamic adjustments that reflect both psychological readiness and physical performance. The system adjusts workout intensity, movement selection, and rest timing in real time based on emotional metrics. The system improves the accuracy of fitness guidance by validating physical coaching decisions with emotional indicators, such as stress levels, mood stability, and energy patterns. The system also strengthens emotional interpretations by comparing emotional indicators with movement quality and biometric data. The system calculates numerical thresholds across these multimodal signals and uses these thresholds to drive coaching adjustments that a human cannot produce in real time. This combined capability creates a unified model that improves the technical performance of adaptive personal computing systems.

In various aspects of the present disclosure, the term real time refers to system behavior that produces an output within a time window that aligns with the user's immediate activity. A real-time system receives sensor input, evaluates the input with machine learning models, and delivers feedback fast enough for the user to rely on that feedback during the ongoing action. The system processes incoming data continuously or at short intervals that match the user's movement or speech pace. The system does not wait for batch processing or long server cycles. The system instead updates its analysis as new signals arrive, such that the user experiences guidance, corrections, and/or insights during the activity itself.

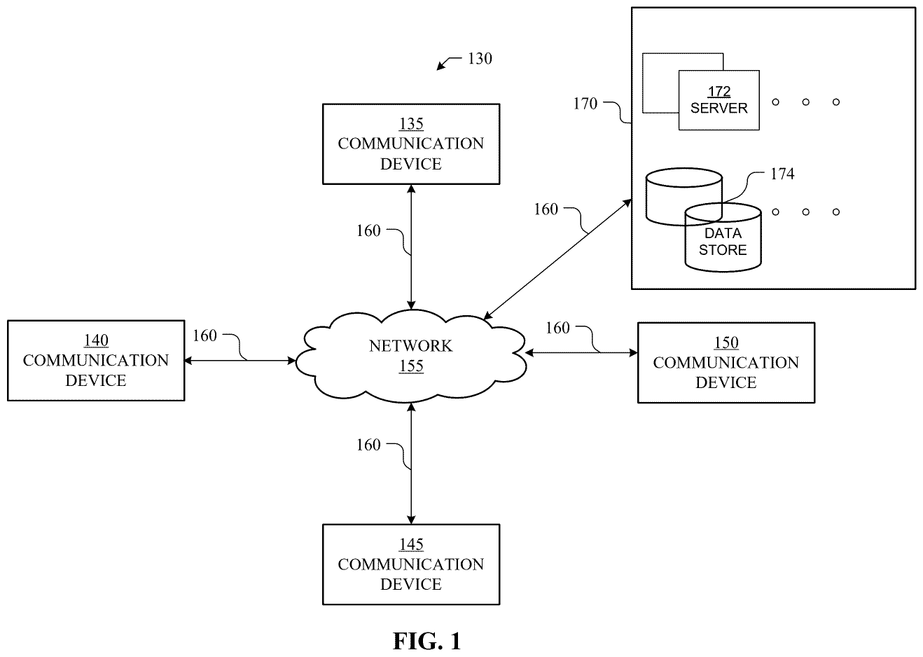

FIG. 1 is a block diagram illustrating an example of a system 130, in accordance with various aspects of the present disclosure. As shown in FIG. 1, the system 130 may include one or more communication devices 135, 140, 145, and 150 and a network device 170. Additionally, the system 130 may include any suitable network, such as, for example, network 155. In some examples, the network 155 may be a Metaverse network. In some examples, the network 155 may be any suitable network capable of provisioning content and/or facilitating communications among entities within, or associated with the network. As an example and not by way of limitation, one or more portions of network 155 may include an ad hoc network, an intranet, an extranet, a virtual private network (VPN), a local area network (LAN), a wireless LAN (WLAN), a wide area network (WAN), a wireless WAN (WWAN), a metropolitan area network (MAN), a portion of the Internet, a portion of the Public Switched Telephone Network (PSTN), a cellular telephone network, or a combination of two or more of these. Network 155 may include one or more networks 155.

Links 160 may connect the communication devices 135, 140, 145, and 150 to network 155, network device 170, and/or to each other. This disclosure contemplates any suitable links 160. In some exemplary embodiments, one or more links 160 may include one or more wireline (such as, for example, Digital Subscriber Line (DSL) or Data Over Cable Service Interface Specification (DOCSIS)), wireless (such as, for example, Wi-Fi or Worldwide Interoperability for Microwave Access (WiMAX)), or optical (such as, for example, Synchronous Optical Network (SONET) or Synchronous Digital Hierarchy (SDH)) links. In some exemplary embodiments, one or more links 160 may each include an ad hoc network, an intranet, an extranet, a VPN, a LAN, a WLAN, a WAN, a WWAN, a MAN, a portion of the Internet, a portion of the PSTN, a cellular technology-based network, a satellite communications technology-based network, another link 160, or a combination of two or more such links 160. Links 160 need not necessarily be the same throughout system 130. One or more first links 160 may differ in one or more respects from one or more second links 160.

Links 160 may connect the communication devices 135, 140, 145, and 150 to network 155, network device 170, and/or to each other. This disclosure contemplates any suitable links 160. In some exemplary embodiments, one or more links 160 may include one or more wireline (such as, for example, Digital Subscriber Line (DSL) or Data Over Cable Service Interface Specification (DOCSIS)), wireless (such as, for example, Wi-Fi or Worldwide Interoperability for Microwave Access (WiMAX)), or optical (such as, for example, Synchronous Optical Network (SONET) or Synchronous Digital Hierarchy (SDH)) links. In some exemplary embodiments, one or more links 160 may each include an ad hoc network, an intranet, an extranet, a VPN, a LAN, a WLAN, a WAN, a WWAN, a MAN, a portion of the Internet, a portion of the PSTN, a cellular technology-based network, a satellite communications technology-based network, another link 160, or a combination of two or more such links 160. Links 160 need not necessarily be the same throughout system 130. One or more first links 160 may differ in one or more respects from one or more second links 160.

Network device 170 may be accessed by the other components of system 130 either directly or via network 155. As an example and not by way of limitation, communication devices 135, 140, 145, 150 may access network device 170 using a web browser or a native application associated with network device 170 (e.g., a mobile social-networking application, a messaging application, another suitable application, or any combination thereof) either directly or via network 155. In particular exemplary embodiments, network device 170 may include one or more servers 172. Each server 172 may be a unitary server or a distributed server spanning multiple computers or multiple datacenters. Servers 172 may be of various types, such as, for example, and without limitation, web server, news server, mail server, message server, advertising server, file server, application server, exchange server, database server, proxy server, another server suitable for performing functions or processes described herein, or any combination thereof. In particular exemplary embodiments, each server 172 may include hardware, software, or embedded logic components or a combination of two or more such components for carrying out the appropriate functionalities implemented and/or supported by server 172. In particular exemplary embodiments, network device 170 may include one or more data stores 174. Data stores 174 may be used to store various types of information. In particular exemplary embodiments, the information stored in data stores 174 may be organized according to specific data structures. In particular exemplary embodiments, each data store 174 may be a relational, columnar, correlation, or other suitable database. Although this disclosure describes or illustrates particular types of databases, this disclosure contemplates any suitable types of databases. Particular exemplary embodiments may provide interfaces that enable communication devices 135, 140, 145, 150, and/or another system (e.g., a third-party system) to manage, retrieve, modify, add, or delete the information stored in data store 174.

Network device 170 may provide users of the system 130 the ability to communicate and interact with other users. In particular exemplary embodiments, network device 170 may provide users with the ability to take actions on various types of items or objects, supported by network device 170. In particular exemplary embodiments, network device 170 may be capable of linking a variety of entities. As an example and not by way of limitation, network device 170 may enable users to interact with each other as well as receive content from other systems (e.g., third-party systems) or other entities, or to allow users to interact with these entities through an application programming interface (API) or other communication channels.

It should be pointed out that although FIG. 1 shows one network device 170 and four communication devices 135, 140, 145, and 150, any suitable number of network devices 170 and communication devices 135, 140, 145, and 150 may be part of the system of FIG. 1 without departing from the spirit and scope of the present disclosure.

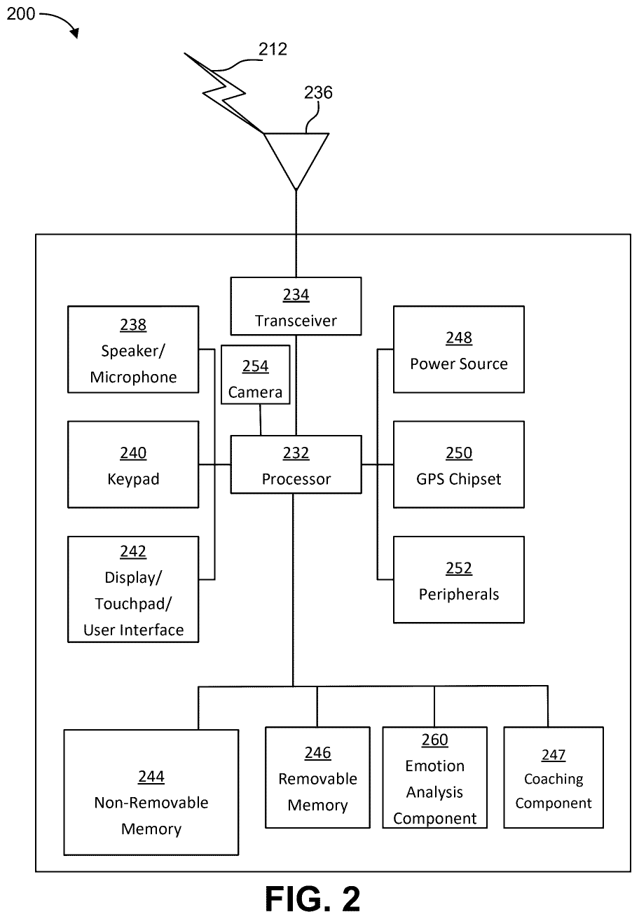

FIG. 2 illustrates a block diagram showing an example hardware and software architecture of a communication device 200, in accordance with various aspects of the present disclosure. The communication device 200 may function as a user equipment (UE), although the device is not limited to any particular form factor. In some implementations, the device 200 may correspond to any of the devices 135, 140, 145, or 150. The device 200 may take the form of a desktop computer, notebook computer, laptop, netbook, tablet computer, e-book reader, GPS unit, digital camera, personal digital assistant, handheld electronic device, cellular phone, smartphone, smart glasses, augmented reality or virtual reality device, head mounted display, smart watch, charging case, or any other suitable electronic device. As shown in FIG. 2, the device 200 includes a processor 232, non-removable memory 244, removable memory 246, a speaker and microphone 238, a keypad 240, a display, touchpad, and user interface 242, a power source 248, a GPS chipset 250, an emotion analysis component 260, a coaching component 247, and peripherals 252. The display, touchpad, and user interface 242 may present content items and receive user interactions. The power source 248 supplies electrical power to the device 200 and may include an AC to DC converter or a USB based charging interface. The device 200 may also include a camera 254. In some implementations, the camera 254 may capture images or video within one or more bounding boxes. The device 200 may further include communication circuitry such as a transceiver 234 and a transmit and receive element 236.

The processor 232 couples to the communication circuitry (including the transceiver 234 and transmit and receive element 236) and executes instructions that control wireless communication with other devices or network nodes. The transmit and receive element 236 may transmit signals to other nodes or may receive incoming signals. For example, the transmit and receive element 236 may function as an antenna that supports radio frequency signaling across a wireless local area network, a wireless personal area network, a cellular network, or other communication technologies. In some implementations, the transmit and receive element 236 may support both radio frequency signaling and light based signaling.

The transceiver 234 may modulate signals for transmission through the transmit and receive element 236 and may demodulate signals received through the transmit and receive element 236. The transceiver 234 may include multiple radio interfaces that support communication across different radio access technologies.

The processor 232 may retrieve data from or store data in the non-removable memory 244 or the removable memory 246. The non-removable memory 244 may include RAM, ROM, flash storage, magnetic drives, or other memory technologies. The removable memory 246 may include secure digital cards, memory sticks, subscriber identity module cards, or other removable storage. In some implementations, the processor 232 may also access memory that resides on external systems such as a server or a cloud-based storage system.

The processor 232 receives power from the power source 248 and distributes power to the other components of the device 200. The power source 248 may include rechargeable batteries, solar cells, fuel cells, or other suitable power storage technologies. The processor 232 may also receive location information from the GPS chipset 250. The GPS chipset 250 may generate longitude and latitude coordinates or provide location estimates using any appropriate positioning method.

The emotion analysis component 260 may implement one or more machine learning models that analyze audible communication captured through the speaker and microphone 238. These models may analyze speech, sighs, laughter, tone changes, and other audible cues to determine emotional state or to generate emotional state summaries over selected periods of time. The emotion analysis component 260 may implement pre-trained models, may train models in real time, or may update the models periodically using training data. The component may correlate the user's emotional state with the user's audible interactions and may present summaries, trend graphs, and citations to specific audio segments through the display, touchpad, and user interface 242.

In some implementations, the emotion analysis component 260 may also include a multimodal speech model that interprets emotional tone from captured audio. The model may classify emotions, compute an emotional range over time, and present emotional trends with references to specific speech segments that support the analysis.

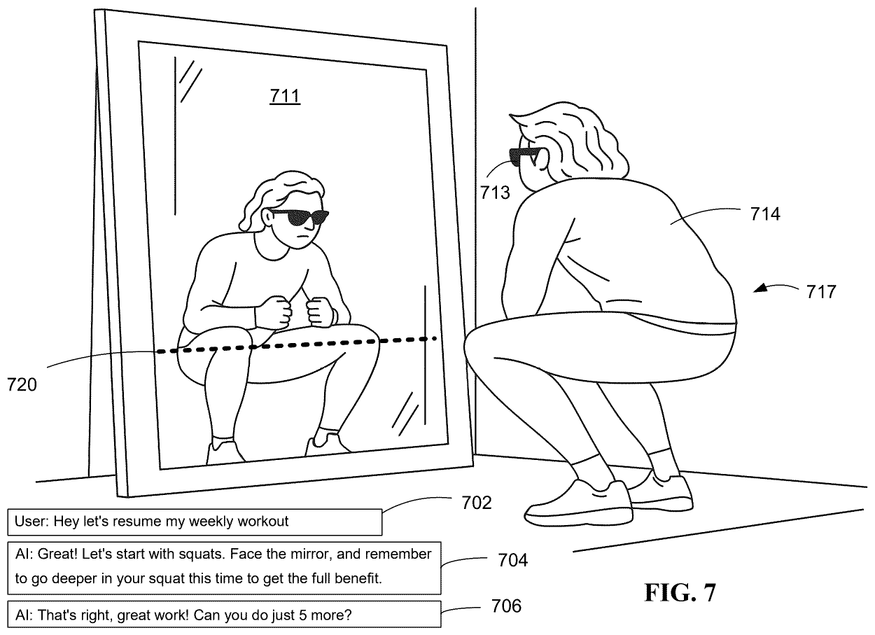

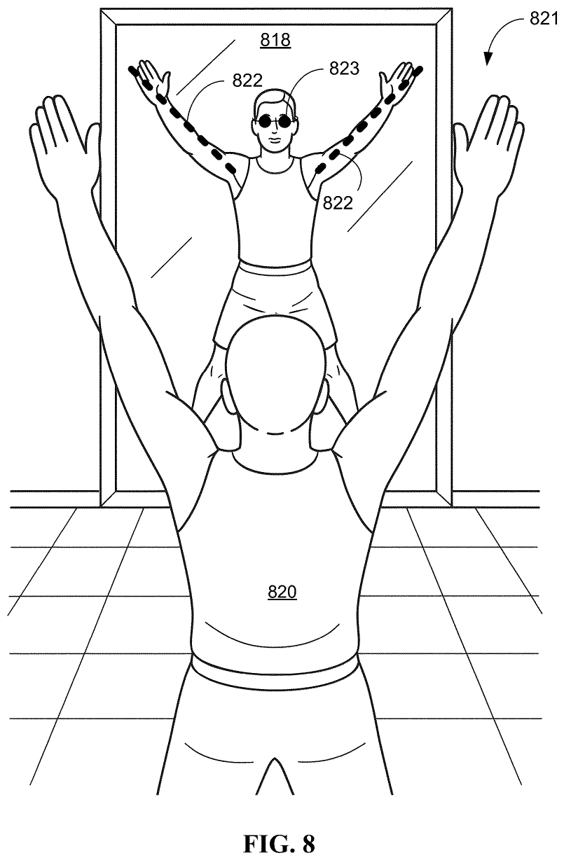

The device 200 may further include a coaching component 247 that evaluates user movement in real time based on image data received through the camera 254. The coaching component 247 may analyze user motion, detect form or posture deviations, and deliver coaching cues that guide the user through physical activities such as exercise routines. In some examples, the coaching component 247 or the coaching component 360, may implement a machine learning model (e.g., machine learning model(s) 930) to provide real-time fitness coaching. In some examples, the coaching component 247 or the coaching component 360 may include an MMAI (e.g., machine learning model(s) 930) configured to perform video analysis. The communication device (e.g., device 200) may be configured to receive a user's (e.g., first user 714 of FIG. 7 or second user 821 of FIG. 8) workout preferences. The device 200 may utilize the coaching component 247 or the coaching component 360 to create/generate one or more workout routines. In an example, a user may provide (e.g., by speaking) one or more workout preferences (e.g., “Hey, let's resume my weekly workout” as illustrated in FIG. 7) to a device (e.g., device 200). In an example, the user may utilize a speaker/microphone 238 to capture (e.g., the speech content of) the one or more workout preferences to the device 200. In another example, the user may utilize a display/touchpad/user interface 242 to provide one or more text-based command(s) to the device 200. The device 200 may utilize the coaching component 247, or coaching component 360 (e.g., AI) to create one or more workout routines, provided by the AI of the coaching component, based on the received one or more workout preferences. In an example, the device 200 may utilize a coaching component, such as the coaching component 247 or the coaching component 360, to provide one or more workout routines to the user. In an example, the device 200 may utilize a speaker/microphone 238 to provide the instruction(s) as audio output to the user. In another example, the device 200 may utilize the display/touchpad/user interface(s) 242 to provide one or more text-based instruction(s) to the display/touchpad/user interface(s) 242 for presentation to the user.

In another example aspect, the device 200 may utilize a camera 254 to observe or record one or more physical movements or poses (e.g., first workout 717 or second workout 821). The device 200 may utilize the coaching component to analyze the first workout 717 or the second workout 821. The coaching component may generate one or more instructions based on the observed or recorded first workout 717 or second workout 821. In one example, the device 200 may utilize a speaker/microphone 238 to capture instruction(s) to the user 612 or user 624. In an example aspect, the instruction(s) may include input on exercise form(s) (e.g., advising the user to “go lower in your squats”). In another example aspect, the instruction(s) 615 may include best practices for a workout(s) (e.g., recommending to “do three sets of ten squats”). In another example aspect, the instruction(s) may include encouragement for the user (e.g., expressing to the user that the user is doing a “great job”).

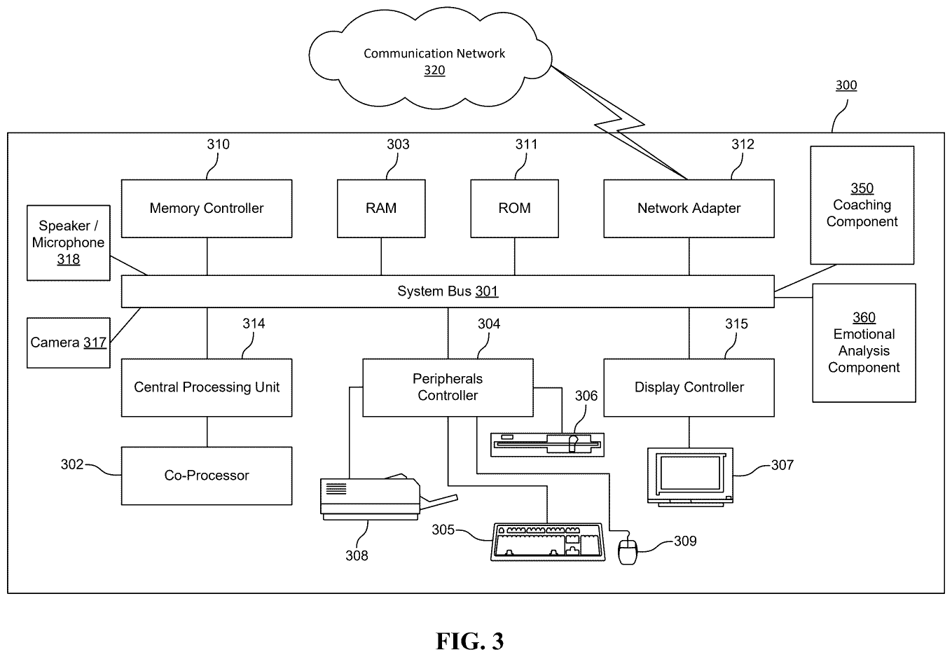

FIG. 3 is a block diagram of an exemplary computing system 300. In some exemplary embodiments, the network device 170 may be a computing system 300. The computing system 300 may include an emotion analysis component 360, and a coaching component 350. The computing system 300 may include a computer or server and may be controlled primarily by computer readable instructions, which may be in the form of software, wherever, or by whatever means such software is stored or accessed. Such computer readable instructions may be executed within a processor, such as central processing unit (CPU) 314, to cause computing system 300 to operate. In many workstations, servers, and personal computers, central processing unit 314 may be implemented by a single-chip CPU called a microprocessor. In other machines, the central processing unit 314 may include multiple processors. Coprocessor 302 may be an optional processor, distinct from main CPU 314, that performs additional functions or assists CPU 314.

In operation, CPU 314 fetches, decodes, and executes instructions, and transfers information to and from other resources via the computer's main data-transfer path, system bus 301. Such a system bus connects the components in computing system 300 and defines the medium for data exchange. System bus 301 typically includes data lines for sending data, address lines for sending addresses, and control lines for sending interrupts and for operating the system bus. An example of such a system bus 301 is the Peripheral Component Interconnect (PCI) bus.

Memories coupled to system bus 301 include RAM 303 and ROM 311. Such memories may include circuitry that allows information to be stored and retrieved. ROMs 311 generally contain stored data that cannot easily be modified. Data stored in RAM 303 may be read or changed by CPU 314 or other hardware devices. Access to RAM 303 and/or ROM 311 may be controlled by memory controller 310. Memory controller 310 may provide an address translation function that translates virtual addresses into physical addresses as instructions are executed. Memory controller 310 may also provide a memory protection function that isolates processes within the system and isolates system processes from user processes. Thus, a program running in a first mode may access only memory mapped by its own process virtual address space; it cannot access memory within another process's virtual address space unless memory sharing between the processes has been set up.

In addition, computing system 300 may contain peripherals controller 304 responsible for communicating instructions from CPU 314 to peripherals, such as printer 308, keyboard 305, mouse 309, and disk drive 306.

The computing system 300 may also include a camera 317. In an exemplary embodiment, the camera 317 may be a smart camera configured to sense images/video appearing within one or more bounding boxes. The computing system 300 may also include a speaker/microphone 318.

Display 307, which is controlled by display controller 315, may be used to display visual output generated by computing system 300. Such visual output may include text, graphics, animated graphics, and video. The display 307 may also include, or be associated with a user interface. The user interface may be capable of presenting one or more content items and/or capturing input of one or more user interactions associated with the user interface. Display 307 may be implemented with a cathode-ray tube (CRT)-based video display, a liquid-crystal display (LCD)-based flat-panel display, gas plasma-based flat-panel display, or a touch-panel. Display controller 315 includes electronic components required to generate a video signal that is sent to display 307.

Further, computing system 300 may contain communication circuitry, such as, for example, a network adaptor 312, that may be used to connect computing system 300 to an external communications network, such as network 155 of FIG. 1, to enable the computing system 300 to communicate with other nodes (e.g., device 200) of the network.

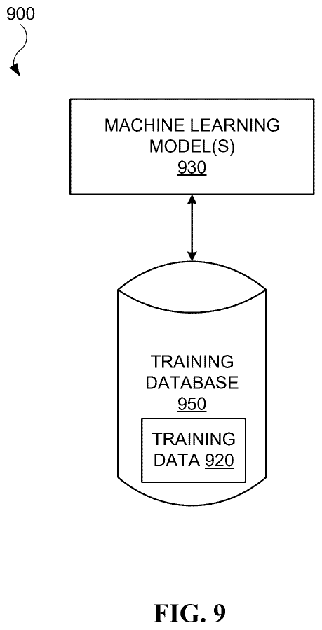

The emotion analysis component 360 may implement a machine learning model(s) (e.g., machine learning model(s) 930 of FIG. 9) to analyze audible communications (e.g., speech, sighs, laughter, tone, etc.) recorded by a device (e.g., computing system 300) to determine the emotional state(s) and/or provide an analysis of the emotional state(s) of a user(s) over time. In some examples, the emotion analysis component 360 may implement a machine learning model(s) (e.g., machine learning model(s) 930 of FIG. 9) and/or an AI model(s) that may be pre-trained, trained in real-time, and/or periodically trained with training data (e.g., training data 920 of FIG. 9) to determine the emotional state(s) of a user(s) (e.g., user 614, user 619, user 620, or user 621) and/or provide the user's emotional state over time based in part on analyzing and/or understanding the audible communications (e.g., speech, sighs, laughter, tone, etc.) of the user over time. The machine learning model(s) (e.g., machine learning model(s) 930 of FIG. 9) may further present a summary of emotional trends over a selected period of time with specific citations (e.g., references to specific speech and/or audible communications) via a display 307.

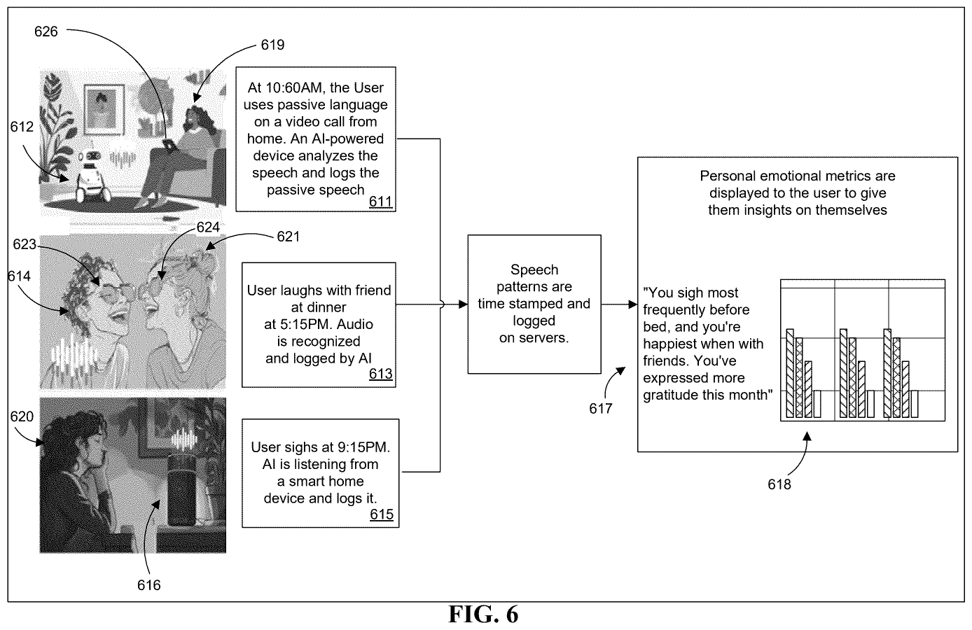

In some examples, the emotion analysis component 360 may further include a multimodal speech model(s) configured to interpret the emotional tone(s) of a user(s), for example, in audio captured/recorded using a device (e.g., computing system 300). In some examples, the emotion analysis component 360 may tally the range of emotions exhibited by the user(s) and/or present a summary of the emotional trends of a user(s) over a given period of time (e.g., emotion trends 618 from FIG. 6). In some examples, the emotion analysis component 360 may be configured to present one or more citations (e.g., citation(s) 617) to specific speech that may be used as a basis for analysis.

The computing system 300 may further include a coaching component 350 configured to analyze a user's motion/movement (e.g., user movements in real time as viewed using a camera (e.g., camera 317) by a device (e.g., computing system 300), and/or provide coaching (e.g., motivation, exercise form(s) to a user(s).

In some examples, the computing system 300 may include the coaching component 350, which may implement machine learning model(s) 930 to provide real-time fitness coaching. In some examples, the coaching component 350 may include an MMAI model configured to perform video analysis. The coaching component 350 may be configured to receive a user's workout preferences. The coaching component 360 may create/generate one or more workout routines. In an example, a user may provide one or more workout preferences to a device (e.g., computing system 300). For example, the user may use a speaker/microphone 318 to capture one or more workout preferences for the computing system 300, as an audio output (e.g., speech by a user). In another example, the user may use a user input interface (e.g., keyboard 305, display 307, or mouse 309) to provide additional text-based workout preferences to the computing system 300. In an example, the computing system 300 may utilize the coaching component 350 to create/generate one or more workout routines based on the received one or more workout preferences. In an example, the computing system 300 may utilize the coaching component 350 to provide one or more workout routines. In an example, the device may utilize a speaker/microphone 318 to provide the one or more workout routines as audio output. In another example, the computing system 300 may utilize the display 307 to provide/present one or more text-based workout routines to a user(s).

In another example aspect, the computing system 300 may utilize a camera 317 to observe and/or capture/record one or more physical movements and/or poses. The computing system 300 may utilize the coaching component 350 to analyze the first workout 717 or the second workout 821. The coaching component 350 may generate one or more based on the observed or captured/recorded workout. In one example, the computing system 300 may utilize a speaker/microphone 318 to provide/capture the one or more instructions to the user. In an example aspect, the coaching component 350 may provide guidance by including input on exercise form(s) (e.g., advising the user to “go lower in your squats”). In another example aspect, the instruction(s) may include best practices for the workout. In another example aspect, the instruction may include encouragement for the user.

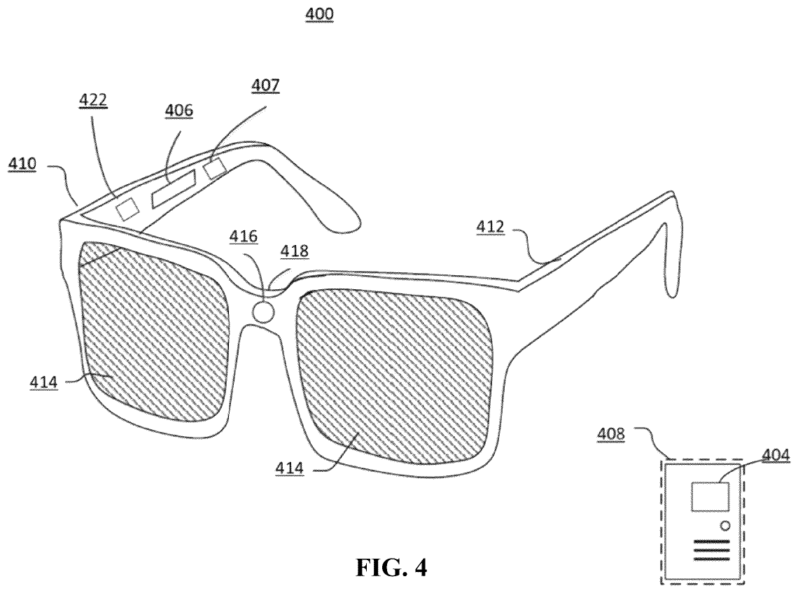

FIG. 4 illustrates an example artificial reality system 400, in accordance with various aspects of the present disclosure. The artificial reality system 400 may include a head-mounted display (HMD) 410 (e.g., smart glasses and/or augmented/virtual reality device) comprising a frame 412, one or more displays 414, a computing device 408 (also referred to herein as computer 408), a controller 404, an emotion analysis component 407, and a coaching component 422. In some examples, the HMD 410 may capture one or more items of text from one or more images/videos associated with a real-world environment in the field of view of one or more cameras (e.g., cameras 416, 418) of the artificial reality system 400. The HMD 410 may utilize the captured text from the one or more images/videos to trigger one or more actions/functions by the artificial reality system 400. The displays 414 may be transparent or translucent, allowing a user wearing the HMD 410 to look through the displays 414 to see the real world (e.g., the real-world environment) and to display visual artificial reality content to the user at the same time. The HMD 410 may include an audio device 406 (e.g., speakers/microphones) that may provide audio artificial reality content to users. The HMD 410 may include one or more cameras 416, 418 which may capture images and/or videos of environments. In one exemplary embodiment, the HMD 410 may include one or more cameras 418 which may be a rear-facing camera tracking movement and/or gaze of a user's eyes.

One of the cameras 416 may be a forward-facing camera capturing images and/or videos of the environment that a user wearing the HMD 410 may view. The camera(s) 416 may also be referred to herein as a front camera(s) 416. The HMD 410 may include an eye tracking system to track the vergence movement of the user wearing the HMD 410. In one exemplary embodiment, the camera(s) 418 may be the eye tracking system. In some exemplary embodiments, the camera(s) 418 may be one camera configured to view at least one eye of a user to capture a glint image(s) (e.g., and/or glint signals). The camera(s) 418 may also be referred to herein as a rear camera(s) 418. The HMD 410 may include a face tracking system to track the muscle movements (e.g., subtle muscle movements) and/or facial expressions/features of the user wearing the HMD 410. In another example aspect of the present disclosure, the camera(s) 418 may be the face tracking system. The camera(s) of the face tracking system may capture one or more images, videos, or the like to track the muscle movements of a user and/or the facial expressions of a user wearing the HMD 410.

The eye tracking system within the HMD 410 may determine pupil dilation(s) by utilizing one or more cameras (e.g., camera(s) 418) and/or other sensors such as scanning systems aimed at an eye(s) of a user(s). The cameras may capture high-resolution images and/or videos of the eye(s) at frequent intervals. In some example aspects, the eye tracking system may utilize image processing applications or image processing algorithms to analyze the captured images and/or videos in real-time to facilitate determination of pupil dilation(s).

The HMD 410 may include a microphone of the audio device 406 to capture voice input from the user. The artificial reality system 400 may further include a controller 404 comprising a trackpad and one or more buttons. The controller 404 may receive inputs from users and relay the inputs to the computer 408. The controller 404 may also provide haptic feedback to one or more users. The computer 408 may be connected to the HMD 410 and the controller 404 through cables or wireless connections. The computer 408 may control the HMD 410 and the controller 404 to provide the augmented reality content to and receive inputs from one or more users. In some example embodiments, the controller 404 may be a standalone controller or integrated within the HMD 410. The computer 408 may be a standalone host computer device, an on-board computer device integrated with the HMD 410, a mobile device, or any other hardware platform capable of providing artificial reality content to and receiving inputs from users. In some exemplary embodiments, the HMD 410 may include an artificial reality system/virtual reality system.

The audio device 406 may capture/record and/or transcribe audible communications (e.g., speech, sighs, laughter, tone, etc.) made by a user(s) (e.g., user 614, user 619, user 620, or user 621 from FIG. 6) over a period of time. In some examples, the user may select the period of time during which the device may capture/record and/or transcribe the user's audible communications for analysis. The emotion analysis component 407 may implement a machine learning model(s) (e.g., machine learning model 930 of FIG. 9) to analyze audible communication (e.g., speech, sighs, laughter, tone, etc.) captured/recorded by a device (e.g., artificial reality system 400) to determine the emotional state(s) and/or provide an analysis of the emotional state(s) of a user(s) over time. In some examples, the emotion analysis component 407 may implement a machine learning model(s) (e.g., machine learning model(s) 930 of FIG. 9) and/or an AI model that may be pre-trained, trained in real-time, and/or periodically trained with training data (e.g., training data 920 of FIG. 9) to determine the emotional state(s) of a user(s) (e.g., user 614, user 619, user 620, or user 621) and/or provide the user's emotional state over time based in part on analyzing or understanding the audible communications (e.g., speech, sighs, laughter, tone, etc.) of the user over time. The machine learning model(s) (e.g., machine learning model(s) 930 of FIG. 9) may further present a summary of emotional trends of a user(s) over a selected period of time with specific citations (e.g., references to specific speech and/or audible communications; citations 617 from FIG. 6) via a display(s) 414.

In some examples, the emotion analysis component 407 may further include a multimodal speech model(s) configured to determine/interpret the emotional tone(s) of a user(s) (e.g., user 614, user 619, user 620, or user 621) in audio captured/recorded by a device (e.g., audio device 406 or artificial reality system 400). In some examples, the emotion analysis component 407 may tally the range of emotions exhibited by a user(s) and/or may present a summary of the emotional trends of a user(s) over a given period of time (e.g., emotion trends 618 from FIG. 6). In some examples, the emotion analysis component 407 may be configured to present one or more citations (e.g., citation(s) 617) to specific speech that may be used as a basis for analysis.

The artificial reality system 400 may further include a coaching component 422 configured to analyze a user's motion/movement (e.g., f as viewed using a camera (e.g., camera(s) 418) on a device (e.g., artificial reality system 400), and/or provide coaching (e.g., motivation, an exercise form(s)) to the user. In some examples, the coaching component 422 may implement a machine learning model(s) 930 to provide real-time fitness coaching. In some examples, the coaching component 422 may include an MMAI model configured to perform video analysis. The artificial reality system 400 may be configured to receive a user's (e.g., user 612 or user 624) workout preferences. The artificial reality system 400 may utilize the coaching component 422 to create/generate one or more workout routines. In an example, the artificial reality system 400 may utilize an audio device 406 to receive one or more workout preferences (e.g., “Hey, let's resume my weekly workout” as illustrated in FIG. 8) from a user, such as a first user 714 described with reference to FIG. 7 or a second user 820 described with reference to FIG. 8. In another example, the user may utilize a controller 404 or display(s) 414 to provide one or more text-based workout preferences to the artificial reality system 400. The artificial reality system 400 may create one or more workout routines based on the received one or more workout preferences. In an example, the artificial reality system 400 may utilize the coaching component 422 to provide the one or more workout routines to a user. In an example, the artificial reality system 400 may utilize the audio device 406 to provide the one or more workout routines as audio output. In another example, the artificial reality system 400 may utilize the display(s) 414 to provide/present one or more text-based workout routines to a user(s).

In another example aspect, the artificial reality system 400 may utilize the front camera 416 to observe and/or capture one or more physical movements and/or poses (e.g., first workout 717 or second workout 821). The artificial reality system 400 may utilize the coaching component 422 to analyze a workout, such as the first workout 717 or the second workout 821 described with reference to FIGS. 7 and 8, respective. The coaching component 422 may generate one or more instructions based on the observed or captured/recorded first workout 717 or second workout 821. In one example, the artificial reality system may utilize an audio device 406 to provide (e.g., based on audio signals/content) the one or more instructions to the user, such as a first user 714 described with reference to FIG. 7 or a second user 820 described with reference to FIG. 8. In an example aspect, the instruction(s) may include input on an exercise form(s) (e.g., advising the first user 714 to “go lower in your squats”). In another example aspect, guidance may be provided by the coaching component 422 to include best practices for a workout (e.g., recommending the first user 714 to “do three sets of ten squats”). In another example aspect, the instruction(s) may include encouragement for the user (e.g., expressing the user is doing a “great job”).

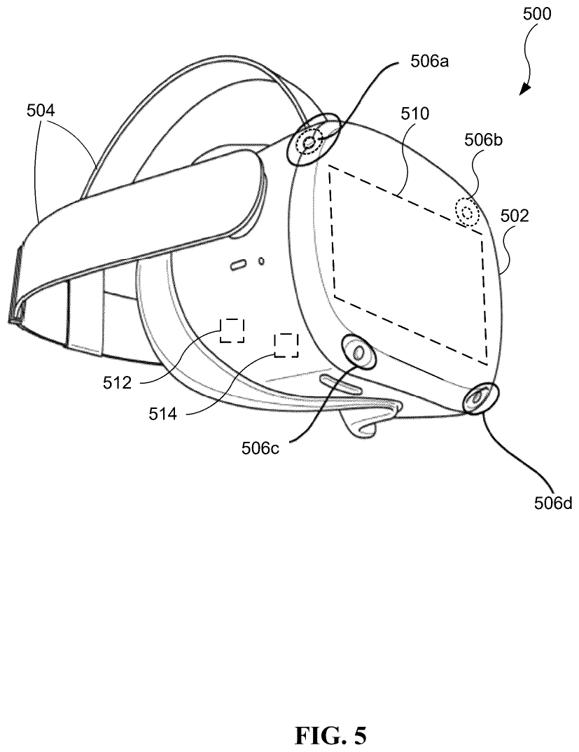

FIG. 5 illustrates an example of an artificial reality system 500, in accordance with various aspects of the present disclosure. In some examples, the artificial reality system 500 may be used for VR applications and/or Augmented Reality (AR)/Mixed Reality (MR) applications. In some examples, the artificial reality system 500 may operate within, or be associated with, a Metaverse network. As shown, the artificial reality system 500 may include an HMD 502. The HMD 502 may include a head strap 504 (also referred to herein as head band) used to fit the HMD 502 onto a user's head. The HMD 502 may further include several image sensors. For example, the HMD 502 may include an image sensor 506a, an image sensor 506b, an image sensor 506c, and an image sensor 506d, and each of the image sensors may be representative of an additional image sensor(s). In some examples, each of the image sensors 506a, 506b, 506c, and 506d may take the form of a camera designed to capture images (e.g., still images, motion images (e.g., video)) of the environment surrounding the HMD 502. Further, in some examples, a compressible shock absorbing device (not shown in FIG. 5) may be mounted on each of the image sensors 506a, 506b, 506c, and 506d. The shock absorbing device may be configured to substantially maintain the structural integrity of the image sensors 506a, 506b, 506c, and 506d in case an impact force is imparted on image sensors 506a, 506b, 506c, and 506d. In some examples, each of the image sensors 506a, 506b, 506c, and 506d may be pivotally and/or translationally mounted to the HMD 502 to pivot the image sensors 506a, 506b, 506c, and 506d at a range of angles and/or to allow for translation in multiple directions, in response to an impact. Also, each of the image sensors 506a, 506b, 506c, and 506d may protrude from a surface (e.g., a front surface, a corner surface, etc.) of the HMD 502 so as to provide the image sensors 506a, 506b, 506c, and 506d with, for example, an increased field of view (e.g., at least 180 degrees field of view), thus allowing the image sensors 506a, 506b, 506c, and 506d to view a relatively greater number of objects (e.g., a hand, a user, a surrounding real-world environment, etc.).

The HMD 502 may further include an assembly 510. In some examples, the assembly 510 may include multiple displays. In this regard, in some examples, the assembly 510 may be referred to as a display assembly or multi-display. As a non-limiting example, the assembly 510 may include an organic light emitting diode (OLED) display, including a micro OLED display. The assembly may be configured to present visual information based on an artificial reality system application(s) (e.g., VR) and/or AR application(s), as well as MR application(s). Additionally or alternatively, the assembly 510 may be coupled (e.g., electrically coupled) to each of the image sensors 506a, 506b, 506c, and 506d, and may present visual information in the form of an external environment, as captured by one or more of the image sensors 506a, 506b, 506c, and 506d.

Additionally, the artificial reality system 500 may include a sensor 512. In one or more implementations, the sensor 512 takes the form of a motion sensor. In this regard, the sensor 512 may take the form of an accelerometer or a gyroscope, as non-limiting examples. The sensor 512 may track motion or movement of the artificial reality system 500. For example, when a user is wearing the artificial reality system 500, the sensor 512 may track the user's head movements.

Additionally, the artificial reality system 500 may include one or more audio transducers 514. The one or more audio transducers 514 may include an audio speaker(s), a microphone(s), or a combination thereof. When the one or more audio transducers 514 include a microphone, the one or more audio transducers 514 may be designed to receive and convert ambient and/or user-based sounds (e.g., a user's spoken words) into electrical signals, and subsequently convert the electrical signals to text.

As discussed above, various aspects of the disclosure use AI to provide feedback on emotional state analysis. In some implementations, the feedback may present the user's emotional state from a third-person perspective. Users often struggle to evaluate their own emotional state from a first-person point of view, and this limitation can reduce self-awareness. Various aspects may use one or more devices to observe the user, interpret emotional cues, and present an analysis that reflects how the user appears from an external vantage point. This approach enhances the user's ability to understand emotional patterns, recognize changes over time, and engage with emotional information that might otherwise be overlooked when relying solely on internal perception.

FIG. 6 illustrates an example system to facilitate emotional state analysis, in accordance with various aspects of the present disclosure. For purposes of illustration, and not of limitation, FIG. 6 may illustrate an example user (e.g., user 619) participating in a video call (e.g., a video call from home). One or more devices (e.g., device 612, device 626) may be configured to record user 619's audible communications during the period of the call. In some examples, device 612 or device 622 may capture/record the user's audible communications during periods other than the video call. In some examples, the device 612 and/or the device 626 may be examples of the device 200, the computing system 300, the artificial reality system 400, or the artificial reality system 500. The 612 and/or the device 626 may capture/record and/or transcribe the user's audible communications. A transcription (e.g., transcription 611) may be generated using an emotion analysis component (e.g., emotion analysis component 260, emotion analysis component 360, or emotion analysis component 407). In an example, the user's speech patterns may be time-stamped and/or logged (e.g., by emotion analysis component 360) on one or more servers (e.g., server(s) 172). In some examples, one or more servers may not be physically located on the device. The emotion analysis component may include a multimodal speech model(s) configured to analyze the user's tone to interpret the user's emotion(s). The emotion analysis component may also be configured to analyze the transcription to determine the user's emotional state(s) and/or provide a summary of the user's emotional state(s) over time. The device may utilize a display (e.g., display/touchpad/user interface(s) 242, display 307, or display(s) 414) to display the user's personal emotional metrics to the user, presenting the user with insights into the user. In an example, the device may use the display to present a summary (e.g., emotional trends 618) of the user's emotional trends over a given period of time (e.g., a day, a month, etc.). In another example, the device may provide one or more citations (e.g., citation(s) 617) to specific speech that may be used as a basis for analysis.

In another example presented in FIG. 6, a group of users (e.g., user 614 and user 621) may be engaged in a conversation while wearing devices (e.g., device 623 or device 624). In this example, device 623 or device 624 may be an artificial reality system (e.g., artificial reality system 400 or 500) configured to capture/record audible communications (e.g., speech, sighs, laughter, tone, other communications) made by a user (e.g., device 623 may be configured to capture/record user 614's audio; device 624 may be configured to capture/record user 261's audio). Consider an example in which device 623 is configured to capture/record and/or transcribe user 614's audible communications in order to analyze the user's emotional state(s). In this example, user 614 or user 621 may be engaged in a conversation. Device 623 may capture/record and/or transcribe user 614's communication during the conversation. Device 623 may recognize the user's (e.g., user 614) audio. Device 623 may utilize an emotion analysis component (e.g., emotion analysis component 260 or 360) to generate a transcription (e.g., transcription 613) based on user 614's audible communications during the conversation. For example, speech patterns may be time-stamped (e.g., transcript 613 may indicate that user 614 laughed at 5:15 PM while at dinner with a friend). In another example, the speech may be logged on one or more servers (e.g., server(s) 172). In some examples, the one or more servers may not be physically located on the device. The emotion analysis component may include a multimodal speech model(s) configured to analyze user 614's tone(s) to interpret the user's emotions. The emotion analysis component may also be configured to analyze the transcription (e.g., transcription 613) to determine the user 614's emotional state and/or provide a summary of the user's emotional state over time. For example, device 623 may use display(s) 414 to display user 614's personal emotional metrics to the user, providing the user with insights associated with the user. In an example, device 623 may use the display(s) 414 to present a summary (e.g., emotional trends 618) of the user's emotional trends over a given period of time. In another example, device 623 may provide one or more citations (e.g., citation(s) 617) to specific speech that may be used as a basis for analysis (e.g., quotes of harsh language used by user 614 may be presented when the device provides an assessment that user 614 was angry).

The transcript indicates that certain implementations extend beyond audio captured during conversations and integrate additional data sources available on wearable or artificial reality devices. For example, devices 623 and 624 may also collect biometric indicators, eye movement signals, or gaze information when available from smart glasses or augmented reality headsets. These devices may monitor pupil dilation, blink frequency, or eye moisture levels to identify subtle emotional cues such as signs of stress or crying. The devices may also analyze how the user interacts with digital content by measuring factors such as the type of posts the user views or likes, the user's screen time patterns, or the rate at which the user switches between applications. These additional inputs allow the emotion analysis component to create a richer representation of emotional state. The emotional analysis component, such as the emotion analysis component 260 or 360, may blend these signals with the transcribed audio to produce a multidimensional emotional profile. This profile may reveal patterns such as increased excitement during certain activities, decreased engagement during specific hours, or a strong positive response during social interactions. Devices 623 and 624, worn by users 614 and 621, can display these patterns and show citations to specific emotional events, such as laughter at a specific time or changes in tone during a sensitive topic.

In another example presented in FIG. 6, a smart home device (e.g., device 616) may be programmed to listen to a user (e.g., user 620) to analyze the user's emotional state over time. In this example, device 616 may be a device 200 or computing system 300 configured to record audible communication (e.g., speech, sighs, laughter, tone) made by user 620. In this example, device 616 may record user 620's audible communications in order to analyze their emotional state. In this example, device 616 may record a sigh made by user 620. Device 616 may utilize an emotion analysis component (e.g., emotion analysis component 260 or emotion analysis component 360) to generate a transcription (e.g., transcription 615) identifying a sigh of the user and/or the time the sigh was made by the user. In another example, the transcription may be logged (e.g., provided by emotion analysis component 260 or 360 to a server) on one or more servers (e.g., server(s) 172). In some examples, one or more servers may not be physically located on the device. The emotion analysis component may include a multimodal speech model(s) configured to analyze user 620's speech (e.g., including the sigh) to interpret the user's emotions. Device 616 may utilize the emotion analysis component to analyze the transcription 615 to determine user 620's emotional state and/or provide a summary of user 620's emotional state(s) over time. In one example, device 616 may send the summary (e.g., emotional trends 618) to another device (e.g., device 200, computing system 300, or artificial reality system 400) for presentation by a display (e.g., display/touchpad/user interface(s) 242, display 307, display(s) 414, or display 930). In this example, the other device may utilize the display to present the summary (e.g., emotional trends 618) of user 620's emotional trends over a given period of time (e.g., a day, a month, etc.). In another example, device 616 may provide an audio presentation of the summary (e.g., emotional trends 618). In another example, device 616 may provide one or more citations (e.g., citation(s) 617) to specific speech that may be used as a basis for analysis (e.g., quotes of harsh language used by user 620 may be presented when the device provides an analysis that user 620 is angry).

In the example of FIG. 6, device 616 illustrates that emotional state analysis may occur even when the user does not actively interact with the device 616. Smart home devices may continuously monitor the environment, detect audible cues such as sighs or changes in speaking style, and record them with corresponding timestamps. Some implementations correlate these audible events with non-audio signals such as changes in lighting conditions, the user's movement through the room, or biometric readings from other devices worn by the user. The emotional analysis component, such as the emotion analysis component 260 or 360, may integrate these signals to identify contextual emotional patterns, such as signs of fatigue at night, heightened stress after certain daily activities, or calm moments during quiet household routines. Implementations may forward these emotional summaries to another device for presentation in a clear and interpretable format. The presentation may include comparisons across days, charts showing frequency of emotional cues, or insights such as increased gratitude over the month or consistent happiness during social interactions.

As discussed above with respect to FIG. 6, emotional state analysis may occur across multiple devices that interact with the user throughout the day. These examples reflect an implementation in which the user generates audible cues in different contexts, such as a video call, an in-person conversation, or an interaction with a smart home device. In these scenarios, one or more devices record the user's speech, interpret emotional tone, and assign timestamps that identify when each emotional cue occurred. The devices may include smart glasses, home speakers, mobile phones, wearable badges, or any device that incorporates a microphone and an emotion analysis component. These implementations collect emotional indicators from scattered moments throughout the day and aggregate them to create an emotional profile. The emotional analysis component may correlate the user's tone with contextual information (e.g., contextual factors) such as location, time of day, and/or interaction type. The device may use these correlations to generate insights such as identifying that the user sighs most frequently before bedtime, that the user laughs more often during social interactions, or that certain times of day consistently reflect higher or lower emotional engagement. These insights may appear on a display to give the user a third-person perspective on their emotional behavior.

In some examples, a user selects the period during which a device records audio that includes speech, sighs, laughter, and other verbal or nonverbal cues. The device implements one or more machine learning models trained on emotional indicators associated with these audible communications. The device uses an emotion analysis component to transcribe the recorded audio and interpret the user's emotional tone through a multimodal speech model. The device then performs sentiment analysis on the transcription and generates a summary of emotional trends over the selected period. The summary may list emotions such as happiness, stress, anger, surprise, and/or calmness, and may correlate these emotions with patterns that extend beyond time of day. For example, an implementation may show that the user laughs more often on certain days, shows improved mood after life events, or expresses more positive emotion during morning routines. The device may also provide citations to specific audio moments that support the emotional interpretation. In some implementations, the device sends the emotional summary to other devices for presentation through a display so the user can view long term emotional trends across different contexts.

As discussed, users often rely on coaching to reach physical fitness goals because a coach may provide structured workouts, technical corrections, and encouragement. Many users cannot access this type of support regularly, and they may struggle to observe their own technique during exercise. Existing fitness technologies may require multiple hardware items, specialized sensors, or heavy headsets, which may feel cumbersome and may limit the user's ability to train comfortably or consistently. Additionally, personal trainers cannot provide the level of precision in guidance, such as correcting a pose and/or body movement. These challenges create a need for a practical approach that uses a single device to observe movement, recommend routines, and provide corrective guidance.

Various aspects of the present disclosure introduce a multimodal AI video analysis approach that may combine several sensing capabilities to deliver real-time fitness coaching through a wearable device or non-wearable device. In some examples, a smart device, such as smart glasses, a smartphone, or a tablet, may capture camera input, inertial measurements, and other sensor data and may use this information to track body movements and poses during exercise. The device may analyze joint angles, limb trajectories, and body alignment in real time and may calculate numerical thresholds that reflect correct form. These thresholds may support guidance that improves precision and responsiveness beyond what a human coach can reliably provide. For example, the device may instruct the user to lower their hips by a specific amount, rotate their torso to reach a particular angle, or raise their arms until their posture matches an optimal reference. Smart glasses may use a mirror to provide a full-body view, and the AI model may combine visual input with motion signals to improve accuracy during fast or complex movements.

Additional implementations may recommend workout routines, monitor the user's progress during the routine, and/or adjust coaching cues based on real-time observations. A device may identify patterns in the user's movement quality, may adapt instruction as fatigue increases, and may introduce variations in technique to improve performance. These approaches may provide a personalized coaching experience that may mitigate the need for specialized gym equipment or continuous access to a human trainer. By observing the user's movements directly and providing detailed feedback on technique, the device may support safer training, more efficient form correction, and improved engagement during exercise.

In some examples, MMAI video technology may track body movements, poses, and/or provide feedback on exercises, such as squats. The AI model may be pre-trained on a database of training data (e.g., training data 920) associated with workouts and/or may have the capability to reference a database of exercises. The database of training data (e.g., training data 920) of workouts may include images, videos, and/or text-based descriptions of exercises. The example exercises may further include descriptions of body movements and/or poses. The training data database may further include example workout routines. This technology may enable fitness coaching via portable devices.

The exemplary aspects of the present disclosure may further enable the use of AI to provide fitness coaching via portable devices, such as wearable devices. A portable device may track body movements and poses, provide fitness coaching through real-time feedback on exercises (e.g., squats, push-ups), and/or recommend workouts generated by AI (e.g., AI model(s)). The AI may provide a workout tailored to an individual's needs. In some examples, a device may be used to track a user's body movements. The AI may provide real-time comments on an exercise form(s) and/or technique(s), or may encourage a user(s) to ensure the user(s) ′ movements are performed safely and correctly. In other examples, MMAI technology may analyze body movements and/or poses of a user(s). An AI coach (e.g., coaching component 247, or coaching component 360, coaching component 350, coaching component 422) may provide specific input on exercise form(s) (e.g., advising the user to “go lower on your squats”) or best practices (e.g., recommending to “do 3 sets of ten squats”, etc.).

FIG. 7 illustrates an example system for real-time coaching using artificial intelligence, in accordance with various aspects of the present disclosure. As shown in the example of FIG. 7, a first user 714 may wear a device 713 while performing a first workout 717. The device 713 may be an example of a UE 100, device 200, computing system 300, an artificial reality system 400, or an artificial reality system 500. The user may provide a command 702 that may include an audible instruction or a text-based workout request, such as “Hey, let us resume my weekly workout.” The device 713 may detect the command 702 and may treat the command 702 as a request to begin or continue coaching.

The device 713 may use a coaching component, such as coaching component 247, 360, or 422, to select or generate a workout routine that aligns with the user's preferences. The coaching component may reference a training database that stores example exercises, variations, and recommended progressions. The device 713 may present instructions through an audio device and may also present visual instructions through display 414 or display and user interface 242. For example, the device 713 may provide feedback 704 that may instruct the user with a statement such as “Great! Let us start with squats. Face the mirror and remember to go deeper in your squat this time to get the full benefit.”

In this example, the first user 714 faces a mirror 711 so the device 713 may capture a full view of the user's posture and alignment. The device 713 may track user movement through one or more cameras, such as camera 254 or front camera 416, and may capture images or video of the user's reflection in the mirror 711. The cameras may observe depth, joint angles, limb movement, and torso alignment during the first workout 717. The device 713 may also combine visual input with motion signals from inertial sensors to increase accuracy when the user moves quickly or changes direction. This multimodal approach may allow the device to maintain precise tracking even when lighting changes, when parts of the body move outside the central frame, or when the user performs compound movements.

The coaching component may analyze the captured movement and may generate real-time instructions that reflect the user's performance. The component may calculate numerical thresholds that define correct form, such as target hip depth, knee-to-toe alignment, torso angle, or foot spacing. The device may provide corrections such as “Lower your hips by a few inches” or “Shift your weight slightly back.” These corrections may include a level of precision and consistency that a human coach cannot provide during live motion. The device may also project a virtual guideline 720 in the user's field of view to show the correct depth or alignment for the movement. The virtual guideline 720 may be an example of a virtual pose correction image. The guideline 720 may update as the user moves, such that the user sees a visual reference that reflects real-time body position. The virtual guideline 720 is not limited to the example illustrated in FIG. 7. The device 713 may present guideline 720 in any form that supports real-time coaching, such as a depth marker, an angle indicator, a posture outline, a joint alignment target, a foot placement boundary, or a target path for movement. The device 713 may also present multiple guidelines simultaneously to support complex exercises. For example, the device 713 may display guideline 720 for hip depth and a second guideline (not shown in FIG. 7) for knee alignment during the first workout 717. The device 713 may display guideline 720 in a manner that makes the guideline appear as if it is drawn directly on mirror 711 or positioned in a virtual space in front of the user so the guideline aligns with the user's reflected body position.

In some examples, the mirror 711 may include one or more cameras, such as camera 254 or camera 416, that capture images or video of the first user 714. These cameras may operate in conjunction with the cameras of the device 713 to increase tracking accuracy, improve pose detection, and provide multiple viewing angles. The mirror 711 may also operate independently of the device 713 and may generate feedback, guideline placement, and analysis when the first user 714 is not wearing the device 713. For example, the mirror 711 may detect the pose of the first user 714 through its integrated cameras, may display one or more guidelines on its surface, and may present coaching cues based on the user's movements.

As the first workout 717 continues, the device 713 may adapt its coaching cues to match the user's movement quality, fatigue level, or pace. The device 713 may provide feedback 706, such as “That's right, great work, can you do 5 more,” or offer best-practice guidance, such as “Keep your chest up and engage your core.” The device 713 may also identify patterns indicating improvement, slowing, or loss of alignment, and may adjust its feedback to support safer and more effective training.

FIG. 8 illustrates an example of a system that facilitates real-time coaching via artificial intelligence, in accordance with various aspects of the present disclosure. A second user 820 may wear a device 823 while performing a second exercise 821, such as a jumping jacks workout. The device 823 may be an example of a UE 100, device 200, computing system 300, an artificial reality system 400, or an artificial reality system 500. The device may use a coaching component, such as coaching component 247, 360, or 422, to generate one or more instructions 622 based on training data and past workouts of the second user 820.

The device 823 may observe the second exercise 821 by tracking the second user's movement through one or more cameras. For example, the device 823 may use camera 254 or front camera 416 to capture images or video of the second user 820 while the user performs the exercise in front of the mirror 818. The mirror 818 may provide a full reflection of the user so that the device 823 can analyze the pose of the second user 820. The pose may include, for example, arm extension, shoulder alignment, leg movement, and/or overall posture during the second exercise 821. In some examples, the mirror 818 may include one or more cameras that operate with or independently of the device 823 to increase visibility and improve motion capture accuracy.

The device 823 may detect the second exercise 821 based on visual and motion patterns and may recognize specific movements through multimodal analysis. Once the device 823 identifies the workout, the coaching component may generate one or more instructions 622 that guide the second user 820. The instructions may include a workout routine, such as “Let us get you warmed up with some jumping jacks. Remember to raise your arms all the way. Let us do a set of twenty, and I will count with you.” The device 823 may also generate guidelines 822 that appear superimposed on to the mirror 818, displayed via the mirror 818, or in a virtual space. Hence, the user sees reference points for correct form. The virtual guidelines 822 may be an example of a virtual pose correction image. Guideline 822 may illustrate ideal arm height, shoulder angle, or limb spacing. In some examples, the device 823 may present multiple guidelines simultaneously to help the second user 820 maintain a consistent movement pattern throughout the second exercise 821.

In some examples, the mirror 818 may include display capabilities and may function as a smart mirror that presents visual content such as guidelines 822, pose outlines, repetition counts, or feedback from the coaching component. The smart mirror 818 may include one or more cameras that observe the user's 820 movements and may display one or more guidelines in positions aligned with the user's reflection. The device 823 may communicate with the smart mirror 818 via wireless signaling, enabling the device and the mirror to share pose information, guideline placement, exercise recognition, and real-time feedback. The smart mirror 818 may supplement the device's observations or may operate independently when the user does not wear the device. In either case, the smart mirror 818 may generate or display coaching cues, movement targets, or technique corrections that assist the user during workout 617.

The device 823 may update the guidelines and instructions in real time as the second user 820 moves. The coaching component may analyze joint angles, limb trajectories, and timing of each repetition and may provide precise corrections that a human coach cannot provide with the same level of accuracy or consistency. The device 823 may encourage the second user with statements such as “Great pace, keep your arms aligned with the guidelines” or “Excellent energy, let us finish this set strong,” while adjusting feedback based on the user's performance.

In accordance with various aspects of the present disclosure, the device 823 may receive one or more workout preferences from a user. The user 820 may provide these preferences through a command, which may include audio input captured by an audio device such as audio device 406 or speaker and microphone 238. The user may also provide a command as text input through controller 404 or display 414. The device 823 may use a coaching component, such as coaching component 247, 360, or 422, to generate one or more workout routines in response to the command. These routines may include instructions 615, such as a recommended set of squats or a structured warm-up sequence.

The device 823 may present instructions to the user through audio output or visual output on display 414 or on the display and user interface 242. The coaching component may then monitor movements and poses of the workout. To do so, the device 823 may use one or more cameras, such as front camera 416 or camera 254, to observe the user directly or through a mirror. The device 823 may record or capture the user's movements and may use the coaching component to analyze each pose of the workout. The coaching component may identify the exercise, compare the movements of the user 820 with reference movements stored in a training database, and recognize when the user 820 begins or transitions between exercises.

The device 823 may generate instructions based on the observed workout. The instructions may include technique corrections, such as advising the user to lower their hips in a squat. The device 823 may also provide best-practice guidance, such as asking the user 820 to complete five additional repetitions. The device 823 may present encouragement to motivate the user, such as stating that the user is doing a great job. These instructions may adapt as the user 820 moves, and the device 823 may modify its feedback when the user accelerates, slows down, or changes form.

The coaching component may provide personalized coaching by referencing training data and using multimodal AI analysis. MMAI video analysis technology may observe a user's movements with high precision and may provide immediate guidance on form and technique. The device 823 may monitor movements directly or through a mirror, and smart glasses may capture each moment of a workout as the user 820 moves. A mobile phone, tablet, or other computing system 300 may observe the user 820 through an integrated camera. The coaching component may analyze the captured images and assess the user's 820 pose and movement quality.

In examples where the device 823 observes the user's 820 reflection, the device 823 may track full-body posture and limb positions through the mirror 811. The coaching component may detect when the user 820 begins exercising and may automatically identify specific exercises based on movement patterns that match examples in the training database 950. The device 823 may use one or more AI models, such as machine learning models 930, that may be pre-trained and may also train in real time using training data 920. These AI models may incorporate exercise manuals, instructional videos, and recorded demonstrations, thereby enhancing the accuracy of form evaluation and technique correction. The coaching component may provide specific technique advice, such as advising the user to go lower during squats or recommending three sets of 10 repetitions. The coaching component may also provide encouragement, learned from training data, to support user motivation throughout the workout.

In some implementations, emotional-state analysis and real-time fitness coaching may operate together within a unified multimodal system. In this combined approach, a device such as a device 200, computing system 300, artificial reality system 400, artificial reality system 500, device 713, or device 823 may monitor the user's daily emotional cues and may also coach the user during physical workouts. This integration may allow the device to tailor workout routines, adjust exercise intensity, adjust the coaching tone, and generate personalized feedback that aligns with the user's emotional state and physical performance.

As described with reference to FIG. 6, a device, such as devices 612, 616, 623, 624, and 626, may record audio across multiple contexts during the user's daily routine. For example, device 626 may capture audio during a video call, device 623 or device 624 may capture laughter or changes in tone during a conversation with a friend, and device 616 may record sighs or quiet utterances when the user spends time at home. These devices may log speech patterns, sighs, laughter, and tone shifts and may generate emotional insights such as “You sigh most frequently before bed” or “You appear happiest when you interact with friends.” The system may use these emotional trends to guide later coaching decisions. For example, if emotional patterns show increased stress in the evening, the device may recommend a calming workout, slower pacing, or additional encouragement during the user's exercise session.

As described with reference to FIG. 7, a device, such as a device 713, may perform precision movement analysis during a workout. For example, while the first user 714 performs a squat, the device 713 may track hip depth, joint angles, and posture in real time. The device 713 may generate guidelines 720 that appear on or near the mirror 711 and may provide corrective cues such as “Go deeper into your squat to get the full benefit.” The device 713 may fuse camera data and motion signals to calculate numerical thresholds that support precise guidance. These thresholds may be combined with the user's emotional trends. For example, if the emotional analysis suggests the first user 714 feels fatigued or discouraged, the device 713 may ease the training load, reduce the number of repetitions, or adjust the tone of feedback to increase motivation. Alternatively, if the emotional analysis suggests the first user 714 is not fatigued or discouraged, the device 713 may increase the training load and/or adjust the tone of the feedback. For example, the device 713 may admonish the user 714 for slacking if the device 713 determines that the user 714 has sufficient energy and engagement to continue the workout with greater effort.

As described with reference to FIG. 8, a device, such as a device 823, may track a second user 820 performing a second exercise 821, such as jumping jacks. The device 823 may observe limb position, shoulder alignment, and repetition timing through one or more cameras and may project guidelines 820 onto the mirror 818 or into a virtual space. The device 823 may adjust these guidelines automatically when emotional cues indicate frustration, stress, or low confidence. For example, if the second user's 820 recent emotional analysis shows increased tension, the device 823 may slow the pace of the jumping jacks or may provide more supportive coaching cues such as “Great pace, stay with me” or “Excellent work, let us finish this set together.”

In combination, the emotion analysis component (e.g., emotion analysis component 260 or 360) and the coaching component (e.g., coaching component 247 or 350) may share data. The emotional analysis component may track changes in sentiment during the workout itself. If the device detects increased strain in the user's tone during exertion, or detects sighing or breath patterns associated with stress, the device may adjust the coaching strategy. The device may switch from instructional cues to encouragement, may modify visual guidelines to simplify the movement, or may shorten an exercise set to support safer and more enjoyable participation.

The combined system may also correlate long-term emotional patterns with long-term fitness behavior. For example, emotional trends from FIG. 6 may reveal that the user expresses more positivity on days when they complete a workout. The device may use this information to propose training times that align with the user's best emotional windows. Likewise, the device may identify times of day when workouts consistently improve mood and may suggest those times in future sessions.

Camera-based implementations described with reference FIGS. 7 and 8 may further benefit from the emotional analysis. A device equipped with cameras may detect subtle facial expressions or posture changes that reflect emotional shifts during training. The device may combine these signals with audio-derived emotional cues to produce a comprehensive assessment of emotional and physical states. Even when the user does not wear the device, the mirror may provide feedback and guidelines that reflect both the user's form and the user's previously analyzed emotional patterns.

This combined approach may create a multimodal adaptive coaching environment where the user receives real-time physical guidance that responds to emotional state, long-term emotional insights that reflect physical activity, and personalized adjustments that support both wellness and performance. The device may continuously refine coaching cues, guideline placement, workout selection, and tone of feedback based on multimodal emotional and physical signals. No human coach can deliver this level of precision, consistency, or continuous emotional-physical integration across the user's entire day.

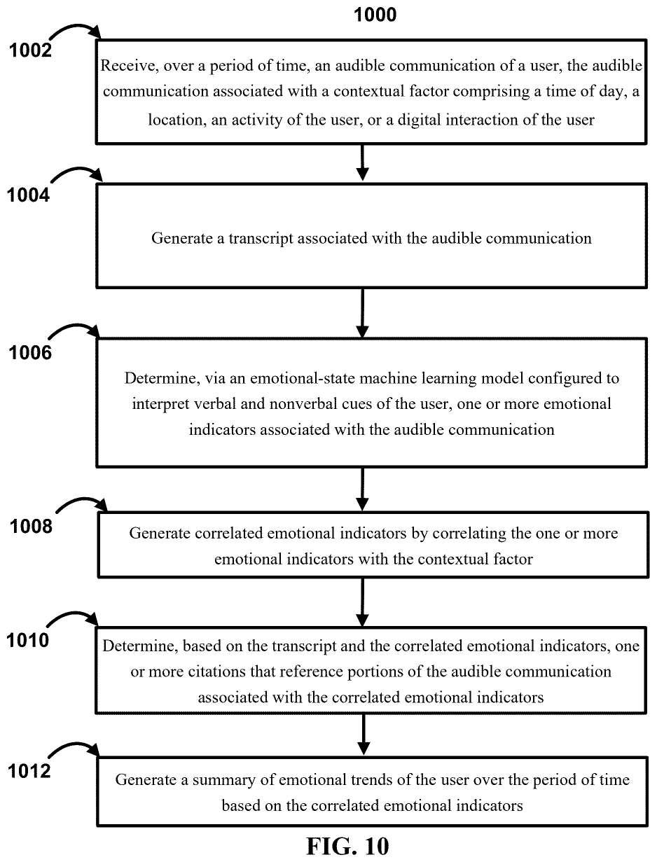

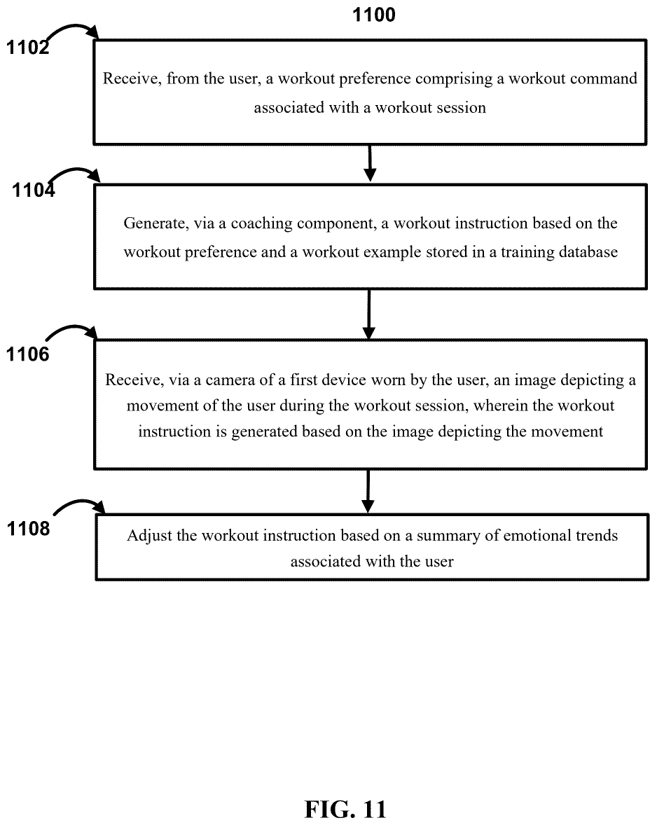

FIG. 9 illustrates an example of a machine learning framework 900 including machine learning model(s) 930 and a training database 950, in accordance with various aspects of the present disclosure. The training database 950 may store training data 920. In some examples, the machine learning framework 900 may be hosted locally in a computing device or hosted remotely. By utilizing the training data 920 of the training database 950, the machine learning framework 900 may train the machine learning model(s) 930 to perform one or more functions, described herein, of the machine learning model(s) 930. In some examples, the machine learning model(s) 930 may be stored in a computing device. For example, the machine learning model(s) 930 may be embodied within a communication device (e.g., device 200). In some other examples, the machine learning model(s) 930 may be embodied within another device (e.g., computing system 300, artificial reality system 400, or artificial reality system 500). Additionally, the machine learning model(s) 930 may be processed by one or more processors (e.g., processor 232 of FIG. 2, coprocessor 302 of FIG. 3, controller 404 of FIG. 4). In some examples, the machine learning model(s) 930 may be associated with operations (or performing operations) of a process 1000 described with reference to FIG. 10 and/or a process 1100 described with reference to FIG. 11. In some other examples, the machine learning model(s) 930 may be associated with other operations. In some examples, the machine learning model(s) 930 may be, or implement, the emotion analysis component 260, the coaching component 247, the emotional analysis component 360 and/or the coaching component 350.

In an example, the training data 920 may include attributes of thousands of objects. For example, the objects may be posters, brochures, billboards, menus, goods (e.g., packaged goods), books, groceries, Quick Response (QR) codes, smart home devices, home or outdoor items, household objects (e.g., furniture, kitchen appliances, etc.) or any other suitable objects. In some other examples, the objects may be smart devices (e.g., devices 200, communication devices 135, 140, 145, 150), persons (e.g., users), newspapers, articles, flyers, pamphlets, signs, cars, content items (e.g., messages, notifications, images, videos, audio), and/or the like. Attributes may include, but are not limited to, the size, shape, orientation, position/location of the object(s), etc. The training data 920 employed by the machine learning model(s) 930 may be fixed or updated periodically. Training data 920 may be updated periodically with data accumulated after/in response to any prior update(s). Alternatively, the training data 920 may be updated in real-time based upon the evaluations performed by the machine learning model(s) 930 in a non-training mode. This may be illustrated by the double-sided arrow connecting the machine learning model(s) 930 and stored training data 920. Some other examples of the training data 920 may include, but are not limited to, one or more emotions associated with audible communications made by one or more users of one or more devices (e.g., users associated with a system (e.g., system 130)). In some examples, audible communications may include speech (e.g., voice data), sighs, laughter, or other nonverbal sounds associated with an expression(s), an emotion(s), or ideas. In some examples, the audible communications may include the tone(s) of a voice of a user while making the communication(s). The audible communication(s) may be analyzed by a device (e.g., computing system 300, device 200, or artificial reality system 400) to determine the emotional state(s) of the user at the time the communication is made. The device may utilize the machine learning model(s) 930 to tally the range of emotions (e.g., happy, sad, angry, fearful, surprised, disgusted, stressed, etc.) presented by one or more users. These audible communications associated with emotions may be provided as a subset of the training data 920 to the training database 950 and may be utilized, in part, to pre-train, and/or train in real-time, the machine learning model(s) 930. Additionally, other content items such as, for example, prior or currently (e.g., in real-time) audible communications associated with one or more emotions of one or more users may be another subset of the training data 920. In this regard, in an instance in which the machine learning model(s) 930 may identify one or more emotions of users in, or associated with, the training data 920 and may determine that the one or more emotions is of a same or similar type as one or more emotions exhibited in communications being analyzed. The machine learning model(s) 930 may automatically identify one or more emotions being exhibited in the communications being analyzed.

In some examples, a component (e.g., emotion analysis component 260 or 360) and/or a device (e.g., device 200, computing system 300, or artificial reality system 400 or 500) may implement the machine learning model(s) 930 to analyze the audible communications of a user(s) to identify one or more emotions of a user(s) during a time period in which the user's communications are being captured/recorded. The device may be capable of listening to and recording audible communications made by a user (e.g., user(s) 214, user(s) 619, user(s) 620, or user(s) 621). Example devices (e.g., device 200, computing system 300, or artificial reality system 400 or 500) may include smart glasses, smartwatches, cellular phones, headphones, laptop computers, or smart home speakers, etc. The machine learning model(s) 930 may include a multimodal speech model(s) configured to capture/record audio and/or interpret the emotional tone(s) of a captured/recorded audible communications. In some examples, the emotion analysis component may quantify a user's emotional state over a period of time selected by the user. A device may utilize the machine learning model(s) 930 to present a summary (e.g., emotional trends 618) of the emotional trends of a user(s) over a given period of time. In some examples a device may utilize the machine learning model(s) 930 to provide specific citations to communications that formed the basis of analysis (e.g., when presenting an analysis of a user(s) being angry, a device may present quotes of harsh language being used).

In some examples, the machine learning model(s) 930 may include a MMAI model configured to perform video analysis. Some other examples of training data 920 may include, but are not limited to, a database of example workout routines and/or example exercises. The example exercises may include images, videos, and/or text-based descriptions of exercises. The example exercises may further include descriptions of body movements and/or poses. The example exercises may be analyzed by a device (e.g., computing system 300, device 200, or artificial reality system 400), to identify one or more exercises that may be observed using a camera 254, camera 317, or front camera 416. The example workout routines may be analyzed by the device to identify and/or generate/create one or more workout routines based on one or more workout preferences (e.g., command(s) 614) that may be received by the device. The categories of exercises and/or workout routines may be provided as a subset of training data 920 to the training database 950 and may be utilized, in part, to pre-train, and/or train in real time, the machine learning model(s) 930. Additionally, other content items such as, for example, prior or currently (e.g., in real-time) workout preferences, workout routines, or exercises may be another subset of training data 920. In this regard, in an instance in which the machine learning model(s) 930 may determine/identify one or more exercises associated with one or more body movements and/or poses in, or associated with, the training data 920 and may determine that a movement(s) and/or pose(s) of the same or similar type is being performed by a user (e.g., user 612 or user 624) being analyzed or captured (e.g., capture of an image(s) and/or video(s)) by the device, the machine learning model(s) 930 may automatically determine the exercise(s) being performed and may provide one or more instruction(s) 615, instruction(s) 616, or instruction(s) 622 to a user(s).

The machine learning model(s) 930 may be trained to generate/create one or more workout routines based on one or more received command(s) 614. In another example, in an instance in which the machine learning model(s) 930 may identify one or more command(s) 614 in, or associated with, the training data 920 and may determine that the one or more workout preferences of the same or similar type is present in one or more workout preferences being analyzed, the machine learning model(s) 930 may create/generate or edit one or more workout routines or may provide one or more instruction(s) 615, instruction(s) 616, or instruction(s) 622 to a user(s). In other examples, the machine learning model(s) 930 may be capable of referencing a database (e.g., training database 950) of workout routines and/or may provide a routine(s) included in the database to a user(s).

In some examples, a coaching component 247, or coaching component 360 and/or device (e.g., device 200, computing system 300, or artificial reality system 400) may implement a machine learning model(s) 930 to receive one or more command(s) 614 from one or more users (e.g., user(s) 612 or user(s) 624). The machine learning model(s) 930 may then generate/create one or more workout routines based on the one or more commands 614. In one example, a device (e.g., device 200, computing system 300, or artificial reality system 400) may utilize a speaker/microphone 238, speaker/microphone 318, or audio device 406 to provide one or more workout routines as audio output to a user. In another example, a device (e.g., device 200, computing system 300, or artificial reality system 400) may utilize a user interface (e.g., display/touchpad/user interface(s) 242, display 307, or display(s) 414) to provide the one or more workout routines using text to a user(s).

In another example aspect, a device (e.g., device 200, computing system 300, or artificial reality system 400) may utilize a camera (e.g., camera 254, camera 317, or front camera 416) to observe and/or capture/record a first workout 717 or second workout 821. The device (e.g., device 200, computing system 300 or artificial reality system 400) may implement the machine learning model(s) 930 to analyze the first workout 717 or second workout 821. In one example aspect, the machine learning model(s) 930 may generate one or more instruction(s) 615, instruction(s) 616, or instruction(s) 622 for provision to a user(s).

FIG. 10 is a flow diagram illustrating an example of a process 1000 for analyzing an emotional state, in accordance with various aspects of the present disclosure. The process 1000 may be implemented by a device, such as a device 200, computing system 300, artificial reality system 400, or artificial reality system 500. The process 1000 begins at block 1002 by receiving, over a period of time, an audible communication of a user, the audible communication associated with a contextual factor comprising a time of day, a location, an activity of the user, and/or a digital interaction of the user. At block 1004, the process 1000 generates a transcript associated with the audible communication. At block 1006, the process 1000 determines, via an emotional-state machine learning model configured to interpret verbal and nonverbal cues of the user, one or more emotional indicators associated with the audible communication. At block 1008, the process 1000 generates correlated emotional indicators by correlating the one or more emotional indicators with the contextual factor. At block 1010, the process 1000 determines, based on the transcript and the correlated emotional indicators, one or more citations that reference portions of the audible communication associated with the correlated emotional indicators. At block 1012, the process 1000 generates a summary of emotional trends of the user over the period of time based on the correlated emotional indicators. The summary of emotional trends may comprise the one or more citations.

FIG. 11 is a flow diagram illustrating an example of a process 1100 for real-time fitness coaching, in accordance with various aspects of the present disclosure. The process 1100 may be implemented by a device, such as a device 200, computing system 300, artificial reality system 400, or artificial reality system 500. In some examples, the process 1100 may be implemented by a user to generate/create a workout plan and/or to receive guidance from the device while working out. The process 1100 begins at block 1102 by receiving, from the user, a workout preference comprising a workout command associated with a workout session. At block 1104, the process 1100 generates, via a coaching component, a workout instruction based on the workout preference and a workout example stored in a training database. At block 1106, the process 1100 receives, via a camera of a first device worn by the user, an image depicting a movement of the user during the workout session, wherein the workout instruction is generated based on the image depicting the movement. At block 1108, the process 1100 adjusts the workout instruction based on a summary of emotional trends associated with the user. In some examples, the process 1100 may generate a virtual pose correction image as part of the workout instruction. Additionally, the process 1100 may display the virtual pose correction image at the first device worn by the user or at a second device that mirrors an image of the user. In some examples, the process 1100 may output audio feedback associated with the workout instruction. In some examples, one or more elements of the process 1100 may be combined with one or more elements of the process 1000.

While the disclosed systems have been described in connection with the various examples of the various figures, it is to be understood that other similar implementations may be used or modifications or additions may be made to the described examples of the disclosed real-time coaching components, among other things as disclosed herein. For example, one skilled in the art will recognize that the disclosed real-time coaching method, among other things as disclosed herein in the instant application may apply to any environment, whether wired or wireless, and may be applied to any number of such devices connected via a communications network and interacting across the network. Therefore, the disclosed systems as described herein should not be limited to any single example, but rather should be construed in breadth and scope in accordance with the appended claims.

In describing preferred methods, systems, or apparatuses of the subject matter of the present disclosure - the disclosed real-time coaching method-as illustrated in the Figures, specific terminology is employed for the sake of clarity. The claimed subject matter, however, is not intended to be limited to the specific terminology so selected.

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Some portions of this description describe the embodiments in terms of applications and symbolic representations of operations on information. These application descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as components, without loss of generality. The described operations and their associated components may be embodied in software, firmware, hardware, or any combinations thereof.

Any of the steps, operations, or processes described herein may be performed or implemented with one or more hardware or software components, alone or in combination with other devices. In one embodiment, a software component is implemented with a computer program product comprising a computer-readable medium containing computer program code, which can be executed by a computer processor for performing any or all of the steps, operations, or processes described.

Embodiments also may relate to an apparatus for performing the operations herein. This apparatus may be specially constructed for the required purposes, and/or it may comprise a computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments also may relate to a product that is produced by a computing process described herein. Such a product may comprise information resulting from a computing process, where the information is stored on a non-transitory, tangible computer readable storage medium and may include any embodiment of a computer program product or other data combination described herein.

What is claimed

1. A method for emotional state analysis, comprising:
receiving, over a period of time, an audible communication of a user, the audible communication associated with a contextual factor comprising a time of day, a location, an activity of the user, or a digital interaction of the user;
generating a transcript associated with the audible communication;
determining, via an emotional-state machine learning model configured to interpret verbal and nonverbal cues of the user, an emotional indicator associated with the audible communication;
generating correlated emotional indicators based on correlating the emotional indicator with the contextual factor;
determining, based on the transcript and the correlated emotional indicators, a citation that reference portions of the audible communication associated with the correlated emotional indicators; and
generating a summary of emotional trends of the user over the period of time based on the correlated emotional indicators, the summary of emotional trends comprising the citation.

2. The method of claim 1, wherein the audible communication is recorded at a device over the period of time.

3. The method of claim 1, further comprising:
receiving, from the user, a workout preference comprising a workout command associated with a workout session; and
generating, via a coaching component, a workout instruction based on the workout preference and a workout example stored in a training database.

4. The method of claim 3, further comprising:
receiving, via a camera of a first device worn by the user, an image depicting a movement of the user during the workout session, wherein the workout instruction is based on the image depicting the movement.

5. The method of claim 4, further comprising:
adjusting the workout instruction based on the summary of emotional trends.

6. The method of claim 4, wherein:
the workout instruction comprises a virtual pose correction image; and
the virtual pose correction image is displayed at the first device worn by the user or a second device that mirrors an image of the user.

7. The method of claim 4, wherein the workout instruction comprises audio feedback.

8. An apparatus for emotional state analysis, comprising:
one or more processors; and
one or more memories coupled with the one or more processors and storing processor-executable code that, when executed by the one or more processors, is configured to cause the apparatus to:
receive, over a period of time, an audible communication of a user, the audible communication associated with a contextual factor comprising a time of day, a location, an activity of the user, or a digital interaction of the user;
generate a transcript associated with the audible communication;
determine, via an emotional-state machine learning model configured to interpret verbal and nonverbal cues of the user, an emotional indicator associated with the audible communication;
generate correlated emotional indicators based on correlating the emotional indicator with the contextual factor;
determine, based on the transcript and the correlated emotional indicators, a citation that reference portions of the audible communication associated with the correlated emotional indicators; and
generate a summary of emotional trends of the user over the period of time based on the correlated emotional indicators, the summary of emotional trends comprising the citation.

9. The apparatus of claim 8, wherein the audible communication is recorded at a device over the period of time.

10. The apparatus of claim 8, wherein execution of the processor-executable code further causes the apparatus to:
receive, from the user, a workout preference comprising a workout command associated with a workout session; and
generate, via a coaching component, a workout instruction based on the workout preference and a workout example stored in a training database.

11. The apparatus of claim 10, wherein execution of the processor-executable code further causes the apparatus to:
receive, via a camera of a first device worn by the user, an image depicting a movement of the user during the workout session, wherein the workout instruction is based on the image depicting the movement.

12. The apparatus of claim 11, wherein execution of the processor-executable code further causes the apparatus to:
adjust the workout instruction based on the summary of emotional trends.

13. The apparatus of claim 11, wherein:
the workout instruction comprises a virtual pose correction image; and
the virtual pose correction image is displayed at the first device worn by the user or a second device that mirrors an image of the user.

14. The apparatus of claim 11, wherein the workout instruction comprises audio feedback.

15. A non-transitory computer-readable medium having program code recorded thereon for emotional state analysis, the program code executed by one or more processors and comprising:
program code to receive, over a period of time, an audible communication of a user, the audible communication associated with a contextual factor comprising a time of day, a location, an activity of the user, or a digital interaction of the user;
program code to generate a transcript associated with the audible communication;
program code to determine, via an emotional-state machine learning model configured to interpret verbal and nonverbal cues of the user, an emotional indicator associated with the audible communication;
program code to generate correlated emotional indicators based on correlating the emotional indicator with the contextual factor;
program code to determine, based on the transcript and the correlated emotional indicators, a citation that reference portions of the audible communication associated with the correlated emotional indicators; and
program code to generate a summary of emotional trends of the user over the period of time based on the correlated emotional indicators, the summary of emotional trends comprising the citation.

16. The non-transitory computer-readable medium of claim 15, wherein the audible communication is recorded at a device over the period of time.

17. The non-transitory computer-readable medium of claim 15, wherein the program code further comprises:
program code to receive, from the user, a workout preference comprising a workout command associated with a workout session; and
program code to generate, via a coaching component, a workout instruction based on the workout preference and a workout example stored in a training database.

18. The non-transitory computer-readable medium of claim 17, wherein the program code further comprises:
program code to receive, via a camera of a first device worn by the user, an image depicting a movement of the user during the workout session, wherein the workout instruction is based on the image depicting the movement.

19. The non-transitory computer-readable medium of claim 18, wherein the program code further comprises:
program code to adjust the workout instruction based on the summary of emotional trends.

20. The non-transitory computer-readable medium of claim 18, wherein:
the workout instruction comprises a virtual pose correction image; and
the virtual pose correction image is displayed at the first device worn by the user or a second device that mirrors an image of the user.
